(19) United States
(12) Patent Application Publication
(10) Pub. No.: US 2022/0387525 A1
(43) Pub. Date: Dec. 8, 2022

(54) COMPOSITIONS AND METHODS FOR MICROBIOME MODULATION

(71) Applicants: Ancilia, Inc., New York, NY (US); Washington University, St. Louis, MO (US)

(72) Inventors: Alexandra Sakatos, Bronx, NY (US); Matthew James Cummings, Ashley, New Milton (GB); Andrew Kau, St. Louis, MO (US); Anne Rosen, St. Louis, MO (US); Antonio David Paez Espino, Concord, CA (US)

(21) Appl. No.: 17/761,594

(22) PCT Filed: Sep. 18, 2020
(86) PCT No.: PCT/US2020/051661

§ 371 Date: Mar. 17, 2022

Related U.S. Application Data
(60) Provisional application No. 62/902,327, filed on Sep. 18, 2019.

(51) Int. Cl. A61K 35/747; A61K 35/745; A61K 35/744; A61K 35/742; A61K 35/741; A61K 35/76; A61P 1/14

(52) U.S. Cl. CPC A61K 35/747; A61K 35/745; A61K 35/744; A61K 35/742; A61K 35/741; A61K 35/76; A61P 1/14

(57) ABSTRACT

The present disclosure provides technologies for modulating microbiome of mammalian subjects (e.g., human subjects). The present disclosure, among others, provides therapeutic compositions and methods of using the same, wherein the therapeutic compositions comprising an engineered population of therapeutic bacteria that (i) are non-pathogenic and commensal in a subject to be administered; and (ii) are resistant to one or more target bacteriophages. In some embodiments, such therapeutic compositions can be useful for treatment of subjects suffering from or susceptible to a microbiome-dysfunction-associated disease, disorder, or condition (e.g., inflammatory bowel disease).

### CROSS-REFERENCE TO RELATED APPLICATIONS

This application claims priority to and benefit of U.S. Provisional Patent Application No. 62/902,327, filed Sep. 18, 2019, the contents of which are hereby incorporated by reference herein in their entirety.

### BACKGROUND

Recent research has demonstrated that the mammalian gastrointestinal tract harbors numerous species of beneficial, commensal bacteria. A reduction in abundance and diversity of the commensal bacteria in the human microbiome has been linked to a wide range of diseases, including inflammatory bowel disease, metabolic disorders, allergy, asthma and autism spectrum disorder. Animal studies have validated this further showing that the transfer of disease-associated microbiota to healthy mice can induce disease phenotypes. Garrett, W. S. et al. Cell 131, 33-45 (2007); Ellekilde, M. et al. Sci Rep 4, 5922 (2014); Sharon, G. et al. Cell 177, 1600-1618.e17 (2019). In recent decades, significant effort and resources have been directed towards the development of bacterial therapies to treat diseases associated with dysbiosis of the gut microbiome. These therapies generally aim to replenish populations of beneficial bacteria that are found to be diminished in disease states. However, most probiotics have shown limited benefits in treating chronic conditions, and systematic reviews have reported no effect of probiotics on fecal microbiota composition. Kristensen, N. B. et al. Genome Med 8, 52 (2016).

### SUMMARY

Although the majority of focus to date has been on the bacterial components of the microbiome, bacteriophages, viruses that infect bacteria, make up at least half of the organisms in the microbiome. While existing studies may have suggested that there is an increase in viral populations in certain disease states, bacterial targets of disease-associated viruses are unknown, and the impact of bacteriophages residing, for example, in human gut, on beneficial bacteria, for example, in human gut, has not yet been characterized. Further, it has been proposed that there is a general lack of predatory interactions between phages and bacteria in human gut. Reyes, A. et al. Nature 466, 334-338 (2010); Chehoud, C. et al. MBio 7, e00322 (2016).

The present disclosure, among other things, provides an insight that in contrast to known views in the art, there are predatory interactions between bacteriophages and bacteria in certain human body sites. Such an insight is based in part on the present discovery that bacteriophages reside in human gut are able to deplete populations of healthy, beneficial bacteria. In particular, the present inventor discovered that addition of a viral fraction containing bacteriophages enriched from fecal samples of individuals (e.g., patients with a microbiome dysfunction-associated disease such as, e.g., in some embodiments inflammatory bowel disease (IBD)) depleted species of beneficial bacteria that were also enriched from the same individuals' samples.

Among other things, the present disclosure also provides an insight that bacteriophages that infect Clostridia bacteria are significantly more abundant in patients with IBD, a microbiome-associated disease. Predatory interactions between phage and beneficial bacteria had not yet been investigated in the context of disease. The finding that phages are present in patients with IBD that attack beneficial bacteria provides an insight that the abundance or presence of such phages can contribute to or drive a reduction in populations of beneficial bacteria, e.g., Clostridia bacteria, in patients with a microbiome dysfunction-associated disease, e.g., IBD. Accordingly, the present disclosure further provides an insight that administration of beneficial bacteria is not necessarily effective to treat a microbiome dysfunction-associated disease because the presence of bacteriophages in a patient suffering from a microbiome dysfunction-associated disease are able to deplete such administered bacteria.

Technologies presented herein address the source of one or more problems associated with certain conventional approaches to treatment of microbiome dysfunction-associated diseases that are based on administration of beneficial bacteria. For example, the present invention provides, among other things, therapeutic compositions that consist of or comprise phage-resistant non-pathogenic commensal bacteria, and various methods and/or materials relating thereto including, for example, methods of administering such compositions, for example to treat diseases or disorders related to the microbiome dysfunction.

In some aspects, provided are methods comprising a step of exposing a subject suffering from or susceptible to a microbiome-dysfunction-associated disease, disorder, or condition, to a population of therapeutic bacteria that (i) are non-pathogenic and commensal in the subject and (ii) are resistant to one or more bacteriophages.

In some embodiments, therapeutic bacteria exposed to a subject in need thereof (e.g., a subject suffering from or susceptible to a microbiome-dysfunction-associated disease, disorder, or condition) each comprise a clustered regularly interspaced short palindromic repeats (CRISPR) spacer that targets the one or more bacteriophages. In some embodiments, therapeutic bacteria exposed to a subject in need thereof (e.g., a subject suffering from or susceptible to a microbiome-dysfunction-associated disease, disorder, or condition) each comprise at least one or more mutants of bacteriophage receptor(s) on bacterial cell surface.

In some embodiments, a population of such therapeutic bacteria are exposed to a subject suffering from or susceptible to a gut microbiome-dysfunction-associated diseases, disorders, or conditions. Exemplary gut microbiome-dysfunction-associated diseases, disorders, or conditions include, but are not limited to, inflammatory bowel disease (IBD) or irritable bowel syndrome, Crohn's disease, ulcerative colitis, immunotherapy-related colitis. In some such embodiments, therapeutic bacteria exposed to subject suffering from or susceptible to a gut microbiome-dysfunction-associated disease, disorder, or condition are resistant to one or more bacteriophages, which may be or comprise Caudovirales.

In some embodiments, a step of exposing a subject in need thereof to a population of therapeutic bacteria (e.g., ones as described herein) comprises administering to such a subject a composition comprising a population of therapeutic bacteria (e.g., ones as described herein).

In some embodiments, a step of exposing a subject in need thereof to a population of therapeutic bacteria (e.g., ones as described herein) comprises administering to such a subject a composition comprising a nucleic acid sequence for altering the genome of host commensal bacteria in the subject such that the host commensal bacterial are genetically engineered to become resistant to target bacteriophages. Such a composition is delivered to host commensal bacteria in a subject in need thereof to produce therapeutic bacteria described herein. Methods for delivering a composition comprising a nucleic acid sequence are known in the art; one skilled in the art will understand that, in some embodiments, such a nucleic acid sequence may be delivered by a recombinant bacteriophage, while in some embodiments, such a nucleic acid sequence may be delivered by a vector. In some embodiments, a nucleic acid sequence for altering the genome of host commensal bacteria comprises one or more CRISPR spacers that target one or more target bacteriophages.

In some embodiments involving therapeutic bacteria described herein, bacteriophages to which such therapeutic bacteria are resistant are associated with a microbiome-dysfunction-associated disease. In some embodiments, bacteriophages to which such therapeutic bacteria are resistant are temperate or non-lytic bacteriophages.

In some embodiments involving therapeutic bacteria described herein, a population of such therapeutic bacteria comprises at least one or more (including, e.g., at least two, at least three, at least four, at least five, or more) isolated, purified, or cultured commensal bacteria selected from the group consisting of Bacillus, Bacteroides, Bifidobacterium, Coprococcus, Clostridium, Collinsella, Desulfomonas, Dorea, Escherichia, Eubacterium, Fusobacterium, Gemmiger, Lactobacillus, Lactoccucs, Monilia, Peptostreptococcus, Propionibacterium, Ruminococcus, and combinations thereof. In some embodiments, a population of therapeutic bacteria comprises Bacteroides, Bifidobacterium, Clostridium, Escherichia, Lactobacillus, Lactoccucs, or combinations thereof. In some embodiments, such bacteria may be autologous. In some embodiments, such bacteria may be allogeneic.

Another aspect described herein relates to therapeutic compositions comprising an engineered population of therapeutic bacteria that (i) are non-pathogenic and commensal in a subject to be administered; and (ii) are resistant to one or more target bacteriophages.

In some embodiments, therapeutic bacteria included in a therapeutic composition described herein each comprise one or more CRISPR spacers that target one or more target bacteriophages. In some such embodiments, therapeutic bacteria included in a therapeutic composition described herein are each genetically engineered to express one or more CRISPR spacers that target one or more target bacteriophages.

In some embodiments, therapeutic bacteria included in a therapeutic composition described herein each comprise at least one or more mutants of bacteriophage receptor(s) on bacterial cell surface. In some such embodiments, therapeutic bacteria included in a therapeutic composition described herein are each genetically engineered to express at least one or more mutants of bacteriophage receptor(s) on bacterial cell surface.

In some embodiments, therapeutic bacteria included in a therapeutic composition described herein comprises at least one or more (including, e.g., at least two, at least three, at least four, at least five, or more) isolated, purified, or cultured bacteria selected from the group consisting of Bacillus, Bacteroides, Bifidobacterium, Coprococcus, Clostridium, Collinsella, Desulfomonas, Dorea, Escherichia, Eubacterium, Fusobacterium, Gemmiger, Lactobacillus, Lactoccucs, Monilia, Peptostreptococcus, Propionibacterium, Ruminococcus, and combinations thereof. In some embodiments, therapeutic bacteria included in a therapeutic composition described herein comprises Bacteroides, Bifidobacterium, Clostridium, Escherichia, Lactobacillus, Lactoccucs, or combinations thereof. In some embodiments, such bacteria may be autologous. In some embodiments, such bacteria may be allogeneic.

Technologies provided herein can be useful for treatment and/or prevention of a microbiome-dysfunction-associated disease, disorder, or condition. Accordingly, technologies provided herein are amenable to subjects suffering from or susceptible to a microbiome-dysfunction-associated disease, disorder, or condition. In some embodiments, technologies provided herein are amenable to subjects suffering from or susceptible to a gut microbiome-dysfunction-associated disease, disorder, or condition. Exemplary gut microbiome-dysfunction-associated diseases, disorders, or conditions include, but are not limited to, inflammatory bowel disease (IBD) or irritable bowel syndrome, Crohn's disease, ulcerative colitis, immunotherapy-related colitis. In some embodiments, subjects administered therapeutic bacteria described herein may be previously administered probiotic therapy, fecal microbiota transplantation (FMT), and/or immunotherapy (e.g., colitis-associated immunotherapy).

These, and other aspects encompassed by the present disclosure, are described in more detail below and in the claims.

### BRIEF DESCRIPTION OF THE DRAWINGS

FIGS. 1A-1B are schematics showing an exemplary in vitro assay for identification of bacteriophages and bacterial hosts. FIG. 1A illustrates isolation of bacteria from patients with inflammatory bowel disease (IBD) or healthy patients to prepare bacterial cultures. FIG. 1B illustrates addition of isolate virus-like particles (VLPs) from the same patients to the bacterial cultures in the presence of mitomycin C (an agent to cause induction of temperate phages). As a positive control, E.coli and T7 phage were added to a subset of cultures. Cultures were grown anaerobically for 72 hours. Bacterial and viral fractions of the cultures were then separated for 16s sequencing.

FIGS. 2A-2C are graphs showing predatory interactions between bacteriophages and bacteria in human gut. FIG. 2A show abundance of bacterial populations determined from 16s sequencing. VLP11-15 refers to a group of samples comprising the bacterial composition without addition of phages. VLP21-25 refers to a group of samples comprising the bacterial composition of the cultures with phages added. Addition of phages causes a reduction in Clostridium scindens and Bifidobacterium longum species. VLP31-35 refers to a group of samples comprising the bacterial composition without phages, with mitomycin C added. VLP41-45 refers to a group of samples comprising the bacterial composition with phages and mitomycin C added. The addition of mitomycin C causes a significant alteration of the bacterial communities, indicating the induction of prophages that attack these bacteria. VLP51-55 refers to a group of samples comprising the bacterial community with E. coli spiked in. VLP61-65 refers to a group of samples comprising the bacterial community with E. coli and T7 phage spiked in. E.coli is eliminated in the presence of T7 phage, indicating that the assay is valid. FIGS. 2B-2C show the presence of phages in the gut that deplete “beneficial” gut bacteria: Clostridium scindens (FIG. 2B) and Bifidobacterium longum (FIG. 2C)

FIG. 3 is a schematic showing an exemplary computational approach for identification of bacteriophages and bacterial hosts. Viral sequences are matched to CRISPR spacers from known bacteria hosts to identify putative bacteria hosts.

FIG. 4 is a graph showing phage populations that infect Clostridia bacteria are more abundant in patients with inflammatory bowel disease (IBD). Phage sequences present in individuals with IBD were queried against a curated collection of CRISPR spacer sequences present in a range of gut bacteria.

FIGS. 5A-5C are graphs showing that phage populations that infect various species of strains of Clostridia bacteria are present in the gut of patients with inflammatory bowel disease (IBD). Phage sequences present in individuals with IBD were queried against a curated collection of CRISPR spacer sequences present in a range of gut bacteria.

### CERTAIN DEFINITIONS

Administering: As used herein, the term “administering” or “administration” typically refers to the administration of a composition to a subject to achieve delivery of an agent to the subject. In some embodiments, the agent is, or is included in, a composition. Those of ordinary skill in the art will be aware of a variety of routes that may, in appropriate circumstances, be utilized for administration to a subject, for example a human. For example, in some embodiments, administration may be ocular, oral, parenteral, topical, etc. In many embodiments provided by the present disclosure, administration is oral administration. In some embodiments, administration may involve only a single dose. In some embodiments, administration may involve application of a fixed number of doses. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time. Administration of cells can be by any appropriate route that results in delivery to a desired location in a subject where at least a portion of the delivered cells or components of the cells remain viable.

Associated: Two events or entities are “associated” with one another, as that term is used herein, if the presence, level and/or form of one is correlated with that of the other. For example, a particular entity (e.g., a bacteriophage) or a particular event (e.g., microbiome dysfunction) is considered to be associated with a disease, disorder, or condition if its presence, level and/or activity correlates with incidence of or susceptibility to the disease, disorder, or condition.

Bacteriophage: As used herein, the term “bacteriophage,” synonymous with the term “phage,” has its conventional meaning as understood in the art, i.e., a virus that infects or selectively infects prokaryotes—such as bacteria, and replicates within the prokaryotes. Bacteriophages include wild-type, naturally occurring, isolated, or recombinant bacteriophages. In some embodiments, bacteriophages are specific to a particular genus or species or strain of bacteria.

Bacteriophage-resistant bacteria: As used herein, the term “bacteriophage-resistant” refers to bacterial strains that are partially or completely resistant to one or more bacteriophages. A partially resistant bacterial strain is a bacterial strain that does not invariably defend against or inhibit phage infection. For example, a partially resistant bacterial strain is a bacterial strain that defends against or inhibits at least 60% or more (including, e.g., at least 70%, at least 80%, at least 90%, at least 95% or more) of incidences of phage infection. A completely resistant bacterial strain is a bacterial strain that defends against or inhibit all incidences of phage infection.

Characteristic sequence element: As used herein, the phrase “characteristic sequence element” refers to a genetic sequence element found in a bacteriophage that represents a characteristic portion of that bacteriophage to be targeted by a CRISPR spacer and that distinguishes from a host sequence (e.g., a sequence found in a bacterial cell that is susceptible to a bacteriophage, and/or a sequence found in a subject to be exposed to therapeutic bacteria described herein). In some embodiments, presence of a characteristic sequence element correlates with presence or level of a particular activity or property of a bacteriophage. In some embodiments, presence (or absence) of a characteristic sequence element defines a particular bacteriophage strain as a member (or not a member) of a particular family or group of such bacteriophages. A characteristic sequence element typically comprises at least two monomers (e.g., nucleotides). In some embodiments, a characteristic sequence element includes at least 2, 3, 4, 5, 6, 7, 8, 9, 10, 11, 12, 13, 14, 15, 20, 25, 30, 35, 40, 45, 50, or more monomers (e.g., contiguously linked monomers). In some embodiments, a characteristic sequence element refers to a sequence of a protospacer (e.g., a sequence in a bacteriophage that a CRISPR spacer specifically targets) of a bacteriophage.

Combination therapy: As used herein, the term “combination therapy” refers to those situations in which a subject is simultaneously exposed to two or more therapeutic regimens (e.g., two or more therapeutic agents). In some embodiments, two or more regimens may be administered simultaneously; in some embodiments, such regimens may be administered sequentially (e.g., all “doses” of a first regimen are administered prior to administration of any doses of a second regimen); in some embodiments, such agents are administered in overlapping dosing regimens. In some embodiments, “administration” of combination therapy may involve administration of one or more agent(s) or modality(ies) to a subject receiving the other agent(s) or modality(ies) in the combination. For clarity, combination therapy does not require that individual agents be administered together in a single composition (or even necessarily at the same time), although in some embodiments, two or more agents, or active moieties thereof, may be administered together in a combination composition.

Comparable: As used herein, the term “comparable” refers to two or more agents, entities, situations, sets of conditions, etc., that may not be identical to one another but that are sufficiently similar to permit comparison therebetween so that one skilled in the art will appreciate that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc. to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Commensal: As used herein, the term “commensal” refers to a microbe that is non-pathogenic to a host subject and is part of normal microflora of the host subject. The term “commensal bacteria” refers to a bacterial cell or population of bacterial cells obtained from, and adapted to, or configured for the microbiome of a mammalian subject. Commensal bacteria are adapted to colonize or configured for colonization of a mammalian subject, for example, in bodily excretions (e.g. saliva, mucus, urine, or stool), surfaces (e.g. mucosal gastrointestinal tract, mouth/pharynx/nares, urogenital track, skin, anus/rectum, cheek/mouth, or eye), and are not adapted for or configured for culture in a laboratory environment.

Complementary: As used herein, the term “complementary” is used in reference to oligonucleotide hybridization related by base-pairing rules. For example, the sequence “C-A-G-T” is complementary to the sequence “G-T-C-A.” Complementarity can be partial or total. Thus, any degree of partial complementarity is intended to be included within the scope of the term “complementary” provided that the partial complementarity permits oligonucleotide hybridization. Partial complementarity is where one or more nucleic acid bases is not matched according to the base pairing rules. Total or complete complementarity between nucleic acids is where each and every nucleic acid base is matched with another base under the base pairing rules.

CRISPR spacer: As used herein, a “CRISPR” spacer stands for “Clustered Regularly Interspaced Short Palindromic Repeats” spacer, and refers to a nucleotide sequence that is present between multiple (e.g., two or more) short direct repeats (i.e., CRISPR repeats) of CRISPR arrays, wherein such a nucleotide sequence is corresponding to (e.g., complementary to) a characteristic sequence element of an invading bacteriophage. In some embodiments, a CRISPR spacer is positioned between two identical CRISPR repeats. In some embodiments, a CRISPR spacer is identified by sequence analysis at the DNA stretches positioned between two CRISPR repeats.

Dosage form: Those skilled in the art will appreciate that the term “dosage form” may be used to refer to a physically discrete unit of an agent (e.g., a therapeutic agent comprising a population of therapeutic bacteria, e.g., ones as described herein) for administration to a subject. Typically, each such unit contains a predetermined quantity of agent. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen). Those of ordinary skill in the art appreciate that the total amount of a therapeutic composition or agent administered to a particular subject is determined by one or more attending physicians and may involve administration of multiple dosage forms.

Dosing regimen: Those skilled in the art will appreciate that the term “dosing regimen” may be used to refer to a set of unit doses (typically more than one) that are administered individually to a subject, typically separated by periods of time. In some embodiments, a given agent has a recommended dosing regimen, which may involve one or more doses. In some embodiments, a dosing regimen comprises a plurality of doses each of which is separated in time from other doses. In some embodiments, individual doses are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population.

Effective amount: An “effective amount” is an amount sufficient to elicit a desired biological response, e.g., treating a condition from which a subject may be suffering. As will be appreciated by those of ordinary skill in this art, the effective amount of a composition or an agent (e.g., a population of therapeutic bacteria as described herein) included in the composition may vary depending on such factors as the desired biological endpoint, the physical, chemical, and/or biological characteristics (e.g., pharmacokinetics and/or cell viability) of agents in the composition, the condition being treated, and the age and health of the subject. An effective amount encompasses therapeutic and prophylactic treatment. For example, in treating a microbiome-dysfunction-associated disease or disorder (e.g., dysbiosis), an effective amount may prevent or reduce at least one symptom associated with the microbiome-dysfunction-associated disease or disorder (e.g., dysbiosis). In some embodiments, an effective amount may recreate a healthy balance of microbiota in microbiome of a subject to be treated. In some embodiments, an effective amount may reduce or suppress inflammation in microbiome of a subject to be treated. In some embodiments, an effective amount may reduce or disrupt infection of non-pathogenic commensal bacteria by bacteriophages. Those skilled in the art will appreciate that an effective amount need not be contained in a single dosage form. Rather, administration of an effective amount may involve administration of a plurality of doses, potentially over time (e.g., according to a dosing regimen).

Engineered: In general, the term “engineered” refers to the aspect of having been manipulated by the hand of man. For example, in some embodiments, a population of cells or microorganisms (e.g., bacteria) is considered to be “engineered” if such a population has been manipulated to form an enriched or purified population of desirable cells or microorganisms, for example, therapeutic bacteria as described herein. In some embodiments, a population of cells or microorganisms (e.g., bacteria) is considered to be “engineered” if genetic information of cells or microorganisms (e.g., bacteria) in the population is altered (e.g., new genetic material not previously present has been introduced, for example by transformation, mating, somatic hybridization, transfection, transduction, or other mechanism, or previously present genetic material is altered or removed, for example by substitution or deletion mutation, or by mating protocols). As is common practice and is understood by those in the art, progeny of cells in an engineered population are typically still referred to as “engineered” even though the actual manipulation was performed on a prior entity.

Enriched: As used herein, the term “enriched” refers to an increase in the proportion of one or more components of a composition. For examples, a therapeutic composition described herein is enriched in therapeutic bacteria that are non-pathogenic and commensal in a subject to be administered and are resistant to one or more target bacteriophages. In some such embodiments, a therapeutic composition described herein contains a higher proportion of therapeutic bacteria (e.g., ones described herein) than that of a reference composition (e.g., a fecal sample composition), for example, by at least 10%, including, e.g., at least 20%, at least 30%, at least 40%, at least 50%, at least 60%, at least 70%, at least 80%, at least 90%, or more. In some embodiments, a therapeutic composition described herein contains at least 70% or more, including, e.g., at least 80%, at least 90%, at least 95%, or up to 100% therapeutic bacteria (e.g., ones described herein), relative to all microorganisms present in such a therapeutic composition.

Host: The term “host” is used herein to refer to a subject to be exposed to a population of therapeutic bacteria (e.g., ones described herein) or a therapeutic composition (e.g., ones described herein). In some embodiments, a host is a subject suffering from or susceptible to a microbiome-dysfunction-associated disease, disorder, or condition. In some embodiments, a host is a subject suffering from or susceptible to infection with a particular bacteriophage associated with a disease, disorder, or condition. In some embodiments, a host is a subject in which certain endogenous non-pathogenic commensal bacteria are engineered to become resistant to one or more bacteriophages.

Increased, Induced, or Reduced: As used herein, these terms or grammatically comparable comparative terms, indicate values that are relative to a comparable reference measurement. For example, in some embodiments, an assessed value or property achieved with a therapeutic bacterium may be “increased” relative to that obtained with a comparable reference bacterium (e.g., a bacterium that is not resistant to one or more target bacteriophage). Alternatively or additionally, in some embodiments, an assessed value or property achieved in a subject may be “increased” relative to that obtained in the same subject under different conditions (e.g., prior to or after an event; or presence or absence of an event such as administration of a population of therapeutic bacteria and/or therapeutic composition described herein), or in a different, comparable subject (e.g., in a comparable subject that differs from the subject of interest in prior exposure to a condition, e.g., absence of administration of a population of therapeutic bacteria and/or therapeutic composition described herein, etc.). In some embodiments, comparative terms refer to statistically relevant differences (e.g., that are of a prevalence and/or magnitude sufficient to achieve statistical relevance). Those skilled in the art will be aware, or will readily be able to determine, in a given context, a degree and/or prevalence of difference that is required or sufficient to achieve such statistical significance.

Inhibit: The term “inhibit” or “inhibition” in the context of risk and/or incidence of a microbiome-dysfunction-associated disease or bacteriophage infection is not limited to only total inhibition. Thus, in some embodiments, partial inhibition or relative reduction is included within the scope of the term “inhibition.” In some embodiments, the term refers to a reduction of the risk or incidence of a microbiome-dysfunction-associated disease or bacteriophage infection to a level that is reproducibly and/or statistically significantly lower than an initial or other appropriate reference level, which may, for example, be a baseline level of risk or incidence of a microbiome-dysfunction-associated disease or bacteriophage infection in the absence or prior to administration of a composition described herein. In some embodiments, the term refers to a reduction of the risk or incidence of a microbiome-dysfunction-associated disease or bacteriophage infection to a level that is less than 75%, less than 50%, less than 40%, less than 30%, less than 25%, less than 20%, less than 10%, less than 9%, less than 8%, less than 7%, less than 6%, less than 5%, less than 4%, less than 3%, less than 2%, less than 1%, less than 0.5%, less than 0.1%, less than 0.01%, less than 0.001%, or less than 0.0001% of an initial level, which may, for example, be a baseline level of risk or incidence of a microbiome-dysfunction-associated disease or bacteriophage infection in the absence or prior to administration of a composition described herein.

Isolated or purified: As used herein, the term “isolated” or “purified” refers to a substance and/or entity that has been (1) separated from at least some of the components with which it was associated when initially produced (whether in nature and/or in an experimental setting), and/or (2) designed, produced, prepared, and/or manufactured by the hand of man. In some embodiments, an isolated substance or entity may be enriched; in some embodiments, an isolated substance or entity may be pure. In some embodiments, isolated substances and/or entities may be separated from about 10%, about 20%, about 30%, about 40%, about 50%, about 60%, about 70%, about 80%, about 90%, about 91%, about 92%, about 93%, about 94%, about 95%, about 96%, about 97%, about 98%, about 99%, or more than about 99% of the other components with which they were initially associated. In some embodiments, isolated agents are about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. In some embodiments, as will be understood by those skilled in the art, a substance may still be considered “enriched”, “isolated” or even “pure”, after having been combined with certain other components such as, for example, one or more carriers or excipients (e.g., buffer, solvent, water, etc.); in such embodiments, percent isolation or purity of the substance is calculated without including such carriers or excipients. Those skilled in the art are aware of a variety of technologies for isolating (e.g., enriching or purifying) substances or agents (e.g., using one or more of fractionation, extraction, precipitation, or other separation).

Microbiota: As used herein, the term “microbiota” refers to collective colonization of a body site of a subject by a diversity ensemble of microbes. Human beings have clusters of bacteria in different parts of the body, such as in the surface or deep layers of skin (skin microbiota), the mouth (oral microbiota), the vagina (vaginal microbiota), and so on. See Huttenhower C. et al. “Structure, function and diversity of the healthy human microbiome” Nature (2012) 486:207-14, the entire contents of which are incorporated herein by reference in their entirety for purposes described herein. For example, in some embodiments, microbiota encompasses “gut microbiota” or “gut flora,” which refers to a microbe population residing in the digestive tract of a subject. Such gut microbiota typically contains tens of trillions of microorganisms, including at least 1000 different species of known bacteria with more than 3 million genes (150 times more than human genes). As will be understood by a skilled artisan, certain species of human gut microbiota is common to a population of human subjects, while certain other species may be specific to individuals.

Modulate: The term “modulate” or “modulation,” which is used in the context of modulating microbiome, refers to an entity whose presence or level in a system in which an activity of interest is observed correlates with a change in level and/or nature of that activity as compared with that observed under otherwise comparable conditions when such modulation is absent. In some embodiments, modulation refers to increasing activity and/or level in one or more particular populations of non-pathogenic commensal bacteria in the presence of a population of therapeutic bacteria and/or a therapeutic composition (e.g., ones as described herein) as compared with that observed under otherwise comparable conditions when such a population of therapeutic bacteria and/or therapeutic composition (e.g., ones as described herein) is absent. In some embodiments, modulation refers to decreasing activity and/or level in target bacteriophages that deplete non-pathogenic commensal bacteria in the presence of a population of therapeutic bacteria and/or a therapeutic composition (e.g., ones as described herein) as compared with that observed under otherwise comparable conditions when such a population of therapeutic bacteria and/or therapeutic composition (e.g., ones as described herein) is absent. In some embodiments, modulation refers to direct interaction with a target entity whose activity is of interest. In some embodiments, modulation refers to indirect interaction (i.e., direct interaction with an intermediate agent that interacts with the target entity) with a target entity whose activity is of interest. In some embodiments, modulation refers to a change in level of a target entity of interest; alternatively or additionally, in some embodiments, modulation refers to a change in activity of a target entity of interest without affecting level of the target entity. In some embodiments, modulation refers to a change in both level and activity of a target entity of interest, so that an observed difference in activity is not entirely explained by or commensurate with an observed difference in level.

Mutant: As used herein, the term “mutant” refers to an organism, a cell, or a biomolecule (e.g., a nucleic acid or a protein) that comprises a genetic variation as compared to a reference organism, cell, or biomolecule. For example, a mutant nucleic acid may, in some embodiments, comprise a mutation, e.g., a nucleobase substitution, a deletion of one or more nucleobases, an insertion of one or more nucleobases, an inversion of two or more nucleobases, as, or a truncation, as compared to a reference nucleic acid molecule. Similarly, a mutant protein may comprise an amino acid substitution, insertion, inversion, or truncation, as compared to a reference polypeptide. Additional mutations, e.g., fusions and indels, are known to those of skill in the art. An organism or cell comprising or expressing a mutant nucleic acid or polypeptide is also sometimes referred to herein as a “mutant.” In some embodiments, a mutant comprises a genetic variant that is associated with a loss of function of a gene product. A loss of function may be a complete abolishment of function, e.g., an abolishment of the enzymatic activity of an enzyme, or a partial loss of function, e.g., a diminished enzymatic activity of an enzyme. In some embodiments, a mutant comprises a genetic variant that is associated with a gain of function, e.g., with a negative or undesirable alteration in a characteristic or activity in a gene product. In some embodiments, a mutant is characterized by a reduction or loss in a desirable level or activity as compared to a reference; in some embodiments, a mutant is characterized by an increase or gain of an undesirable level or activity as compared to a reference. In some embodiments, a reference organism, cell, or biomolecule is a wild-type organism, cell, or biomolecule.

Non-pathogenic: As used herein, the term “non-pathogenic” refers to microbes (e.g., bacteria) that are considered harmless and are not associated with a disease, disorder, or condition. In some embodiments, non-pathogenic microbes are beneficial bacteria residing in a certain body site of a subject, e.g., gut or skin. In some embodiments, a non-pathogenic microbe can become an opportunistic pathogen, e.g., in an immune-compromised host. In some embodiments, non-pathogenic bacteria are not Escherichia coli. In some embodiments, non-pathogenic bacteria are not Streptococcus.

Nucleic acid: As used herein, the term “nucleic acid” refers to a polymer of at least three nucleotides. In some embodiments, a nucleic acid comprises DNA. In some embodiments comprises RNA. In some embodiments, a nucleic acid is single stranded. In some embodiments, a nucleic acid is double stranded. In some embodiments, a nucleic acid comprises both single and double stranded portions. In some embodiments, a nucleic acid comprises a backbone that comprises one or more phosphodiester linkages. In some embodiments, a nucleic acid comprises a backbone that comprises both phosphodiester and non-phosphodiester linkages. For example, in some embodiments, a nucleic acid may comprise a backbone that comprises one or more phosphorothioate or 5′-N-phosphoramidite linkages and/or one or more peptide bonds, e.g., as in a “peptide nucleic acid”. In some embodiments, a nucleic acid comprises one or more, or all, natural residues (e.g., adenine, cytosine, deoxyadenosine, deoxycytidine, deoxyguanosine, deoxythymidine, guanine, thymine, uracil). In some embodiments, a nucleic acid comprises on or more, or all, non-natural residues. In some embodiments, a non-natural residue comprises a nucleoside analog (e.g., 2-aminoadenosine, 2-thiothymidine, inosine, pyrrolo-pyrimidine, 3-methyl adenosine, 5-methylcytidine, C-5propynyl-cytidine, C-5propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereof). In some embodiments, a non-natural residue comprises one or more modified sugars (e.g., 2′-fluororibose, ribose, 2′-deoxyribose, arabinose, and hexose) as compared to those in natural residues. In some embodiments, a nucleic acid has a nucleotide sequence that encodes a functional gene product such as an RNA or polypeptide. In some embodiments, a nucleic acid has a nucleotide sequence that comprises one or more introns. In some embodiments, a nucleic acid may be prepared by isolation from a natural source, enzymatic synthesis (e.g., by polymerization based on a complementary template, e.g., in vivo or in vitro, reproduction in a recombinant cell or system, or chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 1 10, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long.

Operatively associated: As used herein, the term “operatively associated” refers to a juxtaposition wherein the components described are in a relationship permitting them to function in their intended manner. For example, a control element “operatively associated” to a functional element is associated in such a way that expression and/or activity of the functional element is achieved under conditions compatible with the control element. In some embodiments, “operatively associated” control elements are contiguous (e.g., covalently linked) with the coding elements of interest; in some embodiments, control elements act in trans to or otherwise at a from the functional element of interest. In the context of a CRISPR system (e.g., a CRISPR-Cas system), a CRISPR spacer that is operatively associated to a Cas (CRISPR-associated) polypeptide in a CRISPR locus is associated in such a way that a functional guide RNA comprising such a CRISPR spacer is generated to interact a Cas polypeptide for cleavage and degradation of a target sequence that is complementary to the CRISPR spacer.

Pharmaceutical composition: As used herein, the term “pharmaceutical composition” refers to a composition in which an active agent (e.g., a population of therapeutic bacteria described herein) is formulated together with one or more pharmaceutically acceptable carriers. In some embodiments, an active agent (e.g., a population of therapeutic bacteria described herein) is present in unit dose amount appropriate for administration in a therapeutic regimen that shows a statistically significant probability of achieving a predetermined therapeutic effect when administered to a relevant population. In some embodiments, a pharmaceutical composition may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue, capsules, powders, etc. In some embodiments, an active agent may be or comprise a therapeutic bacterium (e.g., ones described herein) or a population of therapeutic bacteria (e.g., a population of a single therapeutic bacteria species or a mixture of different therapeutic bacteria species). In some embodiments, an active agent may be or comprise an isolated, purified, or pure population of therapeutic bacteria (e.g., a population of a single therapeutic bacteria species or a mixture of different therapeutic bacteria species). In some embodiments, an active agent may be or comprise a natural product (whether isolated from its natural source or produced in vitro).

Pharmaceutically acceptable: As used herein, the term “pharmaceutically acceptable” which, for example, may be used in reference to a carrier, diluent, or excipient used to formulate a pharmaceutical composition as disclosed herein, means that the carrier, diluent, or excipient is compatible with the other ingredients of the composition and not deleterious to the recipient thereof.

Pharmaceutically acceptable carrier: As used herein, the term “pharmaceutically acceptable carrier” means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be is “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Prevention: The term “prevention”, as used herein, refers to a delay of onset, and/or reduction in frequency and/or severity of one or more symptoms of a particular disease, disorder or condition. In some embodiments, prevention is assessed on a population basis such that an agent is considered to “prevent” a particular disease, disorder or condition if a statistically significant decrease in the development, frequency, and/or intensity of one or more symptoms of the disease, disorder or condition is observed in a population susceptible to the disease, disorder, or condition. In some embodiments, prevention may be considered complete, for example, when onset of a disease, disorder or condition has been delayed for a predefined period of time.

Pure: As used herein, a population of cells is “pure” if it is substantially free of other cells and/or components. For example, a preparation that contains more than about 90% of therapeutic bacteria (e.g., ones described herein) is typically considered to be a pure preparation. In some embodiments, a therapeutic composition is considered to be “pure” if it contains at least 91%, at least 92%, at least 93%, at least 94%, at least 95%, at least 96%, at least 97%, at least 98%, at least 99%, or up to 100% therapeutic bacteria.

Reference: As used herein, the term “reference” describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control. In some embodiments, a reference is a negative control reference; in some embodiments, a reference is a positive control reference.

Polypeptide: The term “polypeptide”, as used herein, typically has its art-recognized meaning of a polymer of at least three amino acids or more. Those of ordinary skill in the art will appreciate that the term “polypeptide” is intended to be sufficiently general as to encompass not only polypeptides having a complete sequence recited herein, but also to encompass polypeptides that represent functional, biologically active, or characteristic fragments, portions or domains (e.g., fragments, portions, or domains retaining at least one activity) of such complete polypeptides. In some embodiments, polypeptides may contain L-amino acids, D-amino acids, or both and/or may contain any of a variety of amino acid modifications or analogs known in the art. Useful modifications include, e.g., terminal acetylation, amidation, methylation, etc. In some embodiments, polypeptides may comprise natural amino acids, non-natural amino acids, synthetic amino acids, and combinations thereof (e.g., may be or comprise peptidomimetics).

Prophylactically effective amount: A “prophylactically effective amount” is an amount sufficient to prevent (e.g., significantly delay onset or recurrence of one or more symptoms or characteristics of, for example so that it/they is/are not detected at a time point at which they would be expected absent administration of the amount) a condition. A prophylactically effective amount of a composition means an amount of therapeutic agent(s), alone or in combination with other agents, that provides a prophylactic benefit in the prevention of the condition. The term “prophylactically effective amount” can encompass an amount that improves overall prophylaxis or enhances the prophylactic efficacy of another prophylactic agent. Those skilled in the art will appreciate that a prophylactically effective amount need not be contained in a single dosage form. Rather, administration of an effective amount may involve administration of a plurality of doses, potentially over time (e.g., according to a dosing regimen).

Sample: As used herein, the term “sample” typically refers to an aliquot of material obtained or derived from a source of interest. In some embodiments, a source of interest is a biological or environmental source. In some embodiments, a source of interest may be or comprise a cell or an organism, such as a microbe, a plant, an animal, or a subject (e.g., a human). In some embodiments, a source of interest is or comprises biological sample. In some embodiments, a biological sample may be or comprise amniotic fluid, aqueous humor, ascites, bile, bone marrow, blood, breast milk, cerebrospinal fluid, cerumen, chyle, chime, ejaculate, endolymph, exudate, feces, gastric acid, gastric juice, lymph, mucus, pericardial fluid, perilymph, peritoneal fluid, pleural fluid, pus, rheum, saliva, sebum, semen, serum, smegma, sputum, synovial fluid, sweat, tears, urine, vaginal secretions, vitreous humour, vomit, and/or combinations or component(s) thereof. In some embodiments, a biological fluid may be or comprise an intracellular fluid, an extracellular fluid, an intravascular fluid (blood plasma), an interstitial fluid, a lymphatic fluid, and/or a transcellular fluid. In some embodiments, a biological fluid may be or comprise a plant exudate. In some embodiments, a biological tissue or sample may be obtained, for example, by aspirate, biopsy (e.g., fine needle or tissue biopsy), swab (e.g., oral, nasal, skin, or vaginal swab), scraping, surgery, washing or lavage (e.g., bronchioalveolar, ductal, nasal, ocular, oral, uterine, vaginal, or other washing or lavage). In some embodiments, a biological sample is or comprises cells obtained from an individual. In some embodiments, a sample is a “primary sample” obtained directly from a source of interest by any appropriate means. In some embodiments, as will be clear from context, the term “sample” refers to a preparation that is obtained by processing (e.g., by removing one or more components of and/or by adding one or more agents to) a primary sample. For example, filtering using appropriate means in the art (e.g. centrifugation and/or a semi-permeable membrane). Such a “processed sample” may comprise, for example certain bacterial fraction or viral fraction isolated from a sample. In some embodiments, a “processed sample” may comprise, for example nucleic acids or proteins extracted from a sample or obtained by subjecting a primary sample to one or more techniques such as amplification or reverse transcription of nucleic acid, isolation and/or purification of certain components, etc.

Subject: A “subject” to which administration is contemplated includes, but is not limited to, a human (i.e., a male or female of any age group, e.g., a pediatric subject (e.g., infant, child, adolescent) or adult subject (e.g., young adult, middle-aged adult, or senior adult)) and/or a non-human animal, for example, a mammal (e.g., a primate (e.g., cynomolgus monkey, rhesus monkey); a domestic animal such as a cow, pig, horse, sheep, goat, cat, and/or dog; and/or a bird (e.g., a chicken, duck, goose, and/or turkey). In certain embodiments, the animal is a mammal (e.g., at any stage of development). In some embodiments, an animal (e.g., a non-human animal) may be a transgenic or genetically engineered animal. In some embodiments, a subject is a mammalian subject (e.g., a human subject). In some embodiments, a subject is a mammalian subject suffering from a dysbiosis-associated disease, disorder, or condition.

Suffering from: An individual who is “suffering from” a disease, disorder, and/or condition has been diagnosed with and/or displays one or more symptoms of a disease, disorder, and/or condition.

Susceptible to: An individual who is “susceptible to” a disease, disorder, and/or condition is one who has a higher risk of developing the disease, disorder, and/or condition than does a member of the general public. In some embodiments, an individual who is susceptible to a disease, disorder and/or condition may not have been diagnosed with the disease, disorder, and/or condition. In some embodiments, an individual who is susceptible to a disease, disorder, and/or condition may exhibit symptoms of the disease, disorder, and/or condition. In some embodiments, an individual who is susceptible to a disease, disorder, and/or condition may not exhibit symptoms of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Symptoms are reduced: As used herein, “symptoms are reduced” when one or more symptoms of a particular disease, disorder or condition is reduced in magnitude (e.g., intensity, severity, etc.) and/or frequency. For purposes of clarity, a delay in the onset of a particular symptom is considered one form of reducing the frequency of that symptom.

Therapeutic bacteria: As used herein, the phrase “therapeutic bacteria” refers to bacteria that, when administered to a subject, has a therapeutic effect and/or elicits a desired biological and/or pharmacological effect. In some embodiments, a therapeutic bacterium or a population of therapeutic bacteria includes non-pathogenic commensal bacteria that can be used to alleviate, ameliorate, relieve, inhibit, prevent, delay onset of, reduce severity of, and/or reduce incidence of one or more symptoms or features of a disease, disorder, and/or condition associated with dysbiosis or microbiome dysfunction.

Therapeutically effective amount: A “therapeutically effective amount” is an amount sufficient to provide a therapeutic benefit in the treatment of a condition, which therapeutic benefit may be or comprise, for example, reduction in frequency and/or severity, and/or delay of onset of one or more features or symptoms associated with the condition. A therapeutically effective amount means an amount of therapeutic agent(s) (e.g., therapeutic bacteria), alone or in combination with other therapies, that provides a therapeutic benefit in the treatment of the condition. The term “therapeutically effective amount” can encompass an amount that improves overall therapy, reduces or avoids symptoms or causes of the condition, or enhances the therapeutic efficacy of another therapeutic agent. Those skilled in the art will appreciate that a therapeutically effective amount need not be contained in a single dosage form. Rather, administration of an effective amount may involve administration of a plurality of doses, potentially over time (e.g., according to a dosing regimen).

Treat: The terms “treatment,” “treat,” and “treating” refer to reversing, alleviating, delaying the onset of, or inhibiting the progress of a “pathological condition” (e.g., a disease, disorder, or condition, including one or more signs or symptoms thereof) described herein, e.g., a disease, disorder, or condition associated with dysbiosis or microbiome dysfunction including, for example inflammatory bowel diseases. In some embodiments, treatment may be administered after one or more signs or symptoms have developed or have been observed. Treatment may also be continued after symptoms have resolved, for example, to delay or prevent recurrence and/or spread.

Variant: As used herein, the term “variant” refers to an entity that shows significant structural identity with a reference entity but differs structurally from the reference entity in the presence or level of one or more chemical moieties as compared with the reference entity. In many embodiments, a variant also differs functionally from its reference entity. In general, whether a particular entity is properly considered to be a “variant” of a reference entity is based on its degree of structural identity with the reference entity. As will be appreciated by those skilled in the art, any biological or chemical reference entity has certain characteristic structural elements. A variant, by definition, is a distinct chemical entity that shares one or more such characteristic structural elements. To give but a few examples, a small molecule may have a characteristic core structural element (e.g., a macrocycle core) and/or one or more characteristic pendent moieties so that a variant of the small molecule is one that shares the core structural element and the characteristic pendent moieties but differs in other pendent moieties and/or in types of bonds present (single vs double, E vs Z, etc.) within the core, a polypeptide may have a characteristic sequence element comprised of a plurality of amino acids having designated positions relative to one another in linear or three-dimensional space and/or contributing to a particular biological function, a nucleic acid may have a characteristic sequence element comprised of a plurality of nucleotide residues having designated positions relative to on another in linear or three-dimensional space. For example, a variant therapeutic bacterium may differ from a reference therapeutic bacterium as a result of one or more structural modifications (e.g., but not limited to, additions, deletions, and/or modifications of chemical moieties) provided that the variant therapeutic bacterium is resistant to a bacteriophage that targets bacteria associated with a disease, disorder, or condition, e.g., when used in a method described herein. In some embodiments, a variant therapeutic bacterium is characterized in that, when assessed in vitro by culturing, after 24 hours or longer (including, e.g., 48 hours, 72 hours, or longer), such a population of variant therapeutic bacteria in the presence of one or more target bacteriophages, cell viability of such variant therapeutic bacteria is at least 60% or more (e.g., including, e.g., at least 70%, at least 80%, at least 90%, at least 95%, at least 98%, or up to 100%) of that observed when a reference therapeutic bacterium is cultured in the presence of one or more target bacteriophages. In some embodiments, a variant therapeutic bacterium is characterized in that, when assessed in vitro by culturing, after 24 hours or longer (including, e.g., 48 hours, 72 hours, or longer), such a population of variant therapeutic bacteria in the presence of one or more target bacteriophages, cell viability of such variant therapeutic bacteria is at least 1.1-fold or more (e.g., including, e.g., at least 1.2-fold, at least 1.3-fold, at least 1.4-fold, at least 1.5-fold, at least 2-fold, or more) of that observed when a reference therapeutic bacterium is cultured in the presence of one or more target bacteriophages. In some embodiments, a variant therapeutic bacterium exhibits at least one physical characteristic that is different from that of a reference therapeutic bacterium. For example, in some embodiments, a variant therapeutic bacterium may have a genetic alteration in a biological pathway as compared to that of a reference therapeutic bacterium. In some embodiments, a variant has 10, 9, 8, 7, 6, 5, 4, 3, 2, or 1 structural modifications as compared with a reference. In some embodiments, a variant has a small number (e.g., fewer than 5, 4, 3, 2, or 1) number of structural modifications. In some embodiments, a variant has not more than 5, 4, 3, 2, or 1 additions or deletions of chemical moieties, and in some embodiments has no additions or deletions, as compared with a reference. In some embodiments, a variant is an entity that can be generated from a reference by chemical manipulation. In some embodiments, a variant is an entity that can be generated through performance of a synthetic process substantially similar to (e.g., sharing a plurality of steps with) one that generates a reference.

Vector: As used herein, the term “vector” refers to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked.

Standard techniques may be used for recombinant DNA, nucleic acid synthesis, e.g., DNA template synthesis and/or RNA synthesis, and tissue culture and transformation (e.g., electroporation, lipofection). Enzymatic reactions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification. See e.g., Sambrook et al., Molecular Cloning: A Laboratory Manual (2nd ed., Cold Spring Harbor Laboratory Press, Cold Spring Harbor, N.Y. (1989)), which is incorporated herein by reference for any purpose.

### DETAILED DESCRIPTION OF CERTAIN EMBODIMENTS

The present disclosure, among other things, provide technologies relating to therapeutic compositions that consist of or comprise phage-resistant bacteria, and various methods and/or materials relating thereto including, for example, methods of administering such compositions, for example to treat diseases or disorders associated with microbiome.

In some embodiments, provided technologies are more useful in treating microbiome-associated diseases or disorders than certain prior technologies including, for example, administration of conventional probiotics and/or beneficial bacteria. For example, the present disclosure appreciates that many such conventional probiotics (bacterial)-based therapies can be less effective or even ineffective in treatment or prevention of diseases or disorders associated with dysbiosis or microbiome dysfunction. Specifically, the present disclosure, among other things, recognizes that bacteriophages reside in human gut are able to infect and deplete populations of healthy, beneficial bacteria residing in human gut suffering from inflammatory bowel disease (IBD). Thus, the present disclosure, among other things, provides an insight that predatory interactions exist between bacteriophages and bacteria in certain human body sites, and, therefore, the presence of bacteriophages that infect and deplete beneficial commensal bacteria can significantly compromise the efficacy of conventional probiotics (bacterial)-based therapies. The present disclosure, among other things, provides technologies, including therapeutic bacteria, compositions, and methods, that solve such problems, including for example by specifically exposing subjects in need thereof to a population of non-pathogenic commensal bacteria that are resistant to one or more target bacteriophages that are associated with dysbiosis or microbiome-dysfunction-associated diseases or disorders.

### I. Therapeutic Bacteria

The present disclosure provides therapeutic bacteria that (i) are non-pathogenic and commensal in a subject to be treated and (ii) are resistant to one or more bacteriophages that target (e.g., selectively target) corresponding non-pathogenic and commensal host bacteria in the subject. Those skilled in the art, reading the present disclosure, will recognize that provided therapeutic bacteria are useful for treatment and/or prevention of dysbiosis or microbiome-associated diseases or disorders.

### A. Non-Pathogenic and Commensal Bacteria

A microbiome may comprise a variety of non-pathogenic and commensal bacterial species, any one of which may be used in accordance with the present disclosure. In some embodiments, the genus and/or species of non-pathogenic commensal bacterial cells may depend on the specificity of bacteriophages (e.g., phage host range). For example, some bacteriophages exhibit tropism for, or preferentially target, specific species of bacteria.

Bacteria are typically small (typical linear dimensions of around 1 micron), non-compartmentalized, with circular DNA and ribosomes of 70S. In some embodiments, non-pathogenic and commensal bacteria include bacteria from subdivisions of Eubacteria and Archaebacteria. Eubacteria can be further subdivided into gram-positive and gram-negative Eubacteria, which depend upon a difference in cell wall structure. Also included herein are those classified based on gross morphology alone (e.g., cocci, bacilli). In some embodiments, non-pathogenic and commensal bacteria are or comprise Gram-negative cells. In some embodiments, non-pathogenic and commensal bacteria are or comprise Gram-positive cells. Non-limiting examples of non-pathogenic and commensal bacteria that are useful in accordance with the present disclosure include bacteria in the groups of Bacillus, Bacteroides, Bifidobacterium, Brevibacteria, Clostridium, Enterococcus, Escherichia coli, Lactobacillus, Lactococcus, Saccharomyces, and Staphylococcus, e.g., Bacillus coagulans, Bacillus subtilis, Bacteroides fragilis, Bacteroides subtilis, Bacteroides thetaiotaomicron, Bifidobacterium bifidum, Bifidobacterium infantis, Bifidobacterium lactis, Bifidobacterium longum, Clostridium butyricum, Enterococcus faecium, Lactobacillus acidophilus, Lactobacillus bulgaricus, Lactobacillus casei, Lactobacillus johnsonii, Lactobacillus paracasei, Lactobacillus plantarum, Lactobacillus reuteri, Lactobacillus rhamnosus, and Lactococcus lactis (Sonnenborn et al., 2009; Dinleyici et al., 2014; U.S. Pat. Nos. 6,835,376; 6,203,797; 5,589,168; 7,731,976.

In some embodiments, non-pathogenic and commensal bacteria used in therapeutic bacteria can be or comprise one or more (e.g., two or more, three or more, four or more, five or more) of the following bacteria: Clostridium symbiosum, Clostridium hathewayi, Clostridium citroniae, Clostridium bolteae, Ruminococcus sp. M-1, Ruminococcus gnavus, Blautia sp. Canine oral taxon 143, Anaerostipes caccae, Clostridium lactatifermentans, Coprobacillus cateniformis, Clostridium ramosum, cf. Clostridium sp. MLGO55, Clostridium innocuum, Eubacterium desmolans, Clostridium orbiscindens, Ruminococcussp. 16442, Anaerotruncus colihominis, Bacteroides dorei, Bifidobacterium pseudolongum subsp. Pseudolongum, Bifidobacterium breve, Clostridium clostridioforme, Clostridium collagenovorans, Clostridium asparagiforme, Clostridium scindens, Clostridium acetireducens, Clostridium algidicarnis, Clostridium paradoxum, Clostridium saccharogumia, Clostridium ramosum JCM1298, Clostridia bacterium UC5.1-1A9, Clostridium asparagiforme, Clostridium cellulosi, Clostridium bolteae, Clostridium citroniae, Clostridium clostridioforme, Clostridium indolis, Clostridium cocleatum, Clostridium innocuum, Clostridium lavalense, Clostridium saccharolyticum, Clostridium scindens, Clostridium symbiosum, Clostridium butyricum, Clostridia bacterium UC5. 1-1A9, Clostridium jeddahense, Clostridium nigeriense, Clostridium neonatale, Clostridium perfringens, Clostridium phoceensis, Clostridium sp. 1_1_41A1FAA, Clostridium sp. 316002/08, Clostridium sp. 7_3_54FAA, Clostridium sp. ATCC BAA-442, Clostridium sp. C105KSO14, Clostridium sp. CL-6, Clostridium sp. D5, Clostridium sp. FS41, Clostridium sp. HGF2, Clostridium sp. IODB-O3, Clostridium sp. KLE 1755, Clostridium sp. L2-50, Clostridium sp. M62/1, Clostridium sp. MSTE9, Clostridium sp. VE202-10, Clostridia bacterium UC5.1-2G4, Clostridia bacterium UC5.1-2H11, Clostridiales bacterium 1_7_47FAA, Clostridiales bacterium JGI 000176CP_D02, Clostridiales bacterium VE202-03, Clostridiales bacterium VE202-06, Clostridiales bacterium VE202-07, Clostridiales bacterium VE202-09, Clostridiales bacterium VE202-15, Clostridiales bacterium VE202-16, Clostridiales bacterium VE202-21, Clostridiales bacterium VE202-26, Clostridiales bacterium VE202-27, Clostridiales bacterium VE202-28, Clostridiales bacterium VE202-29, Clostridium sp. C8, Clostridium sporogenes, Clostridium tyrobutyricum, Clostridium sp. VE202-10, Lachnoclostridium sp. An131, Lachnoclostridium sp. YL32, Lachnospiraceae bacterium 3_1_57FAA_CT1, Lachnospiraceae bacterium 5_1_57FAA, Lachnospiraceae bacterium 6_1_63FAA, Lachnospiraceae bacterium A4, Lachnospiraceae bacterium DJF_VP30, Lachnospiraceae bacterium VE202-23, Lachnospiraceae bacterium VE202-23, Clostridium acetireducens, Clostridium collagenovorans, and combinations thereof.

In some embodiments, non-pathogenic and commensal bacteria are or comprise one or more species of Clostridia. Exemplary species of Clostridia include, but are not limited to Clostridium scindens, Clostridiales, Clostridium symbosium, Clostridiales bacterium, Clostridium phoceensis, Clostridium innocuum, and combinations thereof. In some embodiments, non-pathogenic and commensal bacteria are or comprise Clostridium scindens.

In some embodiments, non-pathogenic and commensal bacteria are or comprise one or more species of Bifidobacteria. In some embodiments, nonpathogenic and commensal bacteria are or comprise Bifidobacterium longum.

In some embodiments, non-pathogenic and commensal bacteria are or comprise one or more species of Lactobacillus, including, but not limited to Lactobacillus gasseri, Lactobacillus acidophilus, Lactobacillus crispatus, Lactobacillus rhamnosus, Lactobacillus reuteri, Lactobacillus plantarum, Lactobacillus casei, Lactobacillus delbrueckii, Lactobacillus helveticus, Lactobacillus brevis, Lactobacillus fermentum, Lactobacillus buchneri, Lactobacillus sakei.

In some embodiments, non-pathogenic and commensal bacteria are or comprise one or more species of Akkermansia. In some embodiments, non-pathogenic and commensal bacteria are or comprise Akkermansia Mucimphila.

In some embodiments, therapeutic bacteria include one or more (e.g., 1, 2, 3, 4, 5, 6, 7, 8, 9, 10, or more) non-pathogenic and commensal bacteria that are found to normally reside in body sites (e.g., mucosal gastrointestinal tract, mouth/pharynx/nares, urogenital track, skin, anus/rectum, cheek/mouth, or eye) of human subjects. In some embodiments, therapeutic bacteria include one or more variants (e.g., 1, 2, 3, 4, 5, 6, 7, 8, 9, 10, or more variants) of non-pathogenic and commensal bacteria that are found to normally reside in body sites (e.g., mucosal gastrointestinal tract, mouth/pharynx/nares, urogenital track, skin, anus/rectum, cheek/mouth, or eye) of human subjects.

In some embodiments, therapeutic bacteria in a population comprises at least one or more (including, e.g., at least two, at least three, at least four, at least five, or more) isolated, purified, or cultured commensal bacteria selected from the group consisting of Bacillus, Bacteroides, Bifidobacterium, Coprococcus, Clostridium, Collinsella, Desulfomonas, Dorea, Escherichia, Eubacterium, Fusobacterium, Gemmiger, Lactobacillus, Lactoccucs, Monilia, Peptostreptococcus, Propionibacterium, Ruminococcus, and combinations thereof. In some embodiments, a population of therapeutic bacteria comprises Bacteroides, Bifidobacterium, Clostridium, Escherichia, Lactobacillus, Lactoccucs, or combinations thereof. In some embodiments, such bacteria may be autologous (e.g., therapeutic bacteria described herein are genetically engineered from bacteria of a subject to be treated that are susceptible to infection by a target bacteriophage). In some embodiments, such bacteria may be allogeneic.

### B. Bacteriophages

Among other things, the present disclosure provides a therapeutic composition of non-pathogenic and commensal bacteria that are established or are otherwise known to be resistant to one or more bacteriophages that would otherwise infect such non-resistant non-pathogenic and commensal bacteria.

Bacteriophages (also known as phages) are typically composed of proteins that encapsulate a DNA or RNA genome, which may encode only a few or hundreds of genes thereby producing virions with relatively simple or elaborate structures. Thus, bacteriophages are among the most common and diverse entities in the biosphere. Phages are classified according to the International Committee on Taxonomy of Viruses (ICTV) considering morphology and the type of nucleic acid (DNA or RNA, single- or double-stranded, linear or circular). About 19 phage families have been recognized so far that infect bacteria and/or archaea (a prokaryotic domain previously classified as archaebacteria). Many bacteriophages are specific to a particular genus or species or strain of bacterial cells.

In some embodiments, non-pathogenic and commensal bacteria used in compositions and/or methods described herein may be resistant to a lytic bacteriophage. A lytic bacteriophage is one that follows the lytic pathway through completion of the lytic cycle, rather than entering the lysogenic pathway. A lytic bacteriophage undergoes viral replication leading to lysis of the cell membrane, destruction of the cell, and release of progeny bacteriophage particles capable of infecting other cells.

In some embodiments, non-pathogenic and commensal bacteria used in compositions and/or methods described herein may be resistant to a lysogenic bacteriophage. A lysogenic bacteriophage is one capable of entering the lysogenic pathway, in which the bacteriophage becomes a dormant, passive part of the cell's genome through prior to completion of its lytic cycle.

In some embodiments, non-pathogenic and commensal bacteria used in compositions and/or methods described herein may be resistant to a temperate bacteriophage. A temperate is a phage that can be lytic or lysogenic. When lysogenic, such a phage typically integrates its nucleic acid into the host cell genome and remains quiescent, replicating only when the host genome replicates. In its lytic or vegetative multiplication phage, the phage nucleic acid excises itself from the host genome, or does not integrate itself into the host cell genome, but rather takes over the protein synthetic machinery of the host cell at the expense of cellular components and causes phage progeny to be assembled. New phage are released from the infected host cell when the cell lyses.

In some embodiments, non-pathogenic and commensal bacteria used in compositions and/or methods described herein are resistant to a bacteriophage that is virulent to a bacterial cell at one point its life cycle after such a bacterial cell is infected.

While non-pathogenic and commensal bacteria that confer resistance against any target bacteriophage (including, e.g., wild type, naturally occurring, isolated or recombinant bacteriophages) may be used in accordance with the present disclosure, in some embodiments, target bacteriophages that are active against (e.g., able of infecting) one or more non-pathogenic commensal bacterial strains of microbiome in a mammalian subject (e.g., human) are of particular interest. By way of example only, in some embodiments, target bacteriophages to which non-pathogenic and commensal bacteria are resistant include, but are not limited to, those bacteriophage capable of infecting bacteria from at least one or more of the following genera: Bacillus, Bacteroides, Bifidobacterium, Clostridium, Collinsella, Coprococcus, Desulfomonas, Dorea, Escherichia (e.g., E. coli), Eubacterium, Fusobacterium, Gemmiger, Monilia, Lactobacillus, Peptostreptococcus, Propionibacterium, Akkermansia, and Ruminococcus.

In some embodiments, therapeutic bacteria described herein are resistant to one or more bacteriophages selected from the group consisting of Caudovirales or Microviridae phages.

In some embodiments, therapeutic bacteria described herein are resistant to one or more bacteriophages present in a gut microbiome of a human subject. For example, in some such embodiments, therapeutic described herein are resistant to one or more bacteriophages, which may be or comprise Caudovirales.

### C. Exemplary Bacteriophage-Resistant Bacteria

Therapeutic bacteria used in accordance with the present disclosure are resistant to one or more bacteriophages (e.g., ones described herein). In some embodiments, therapeutic bacteria are resistant to one or more bacteriophages that would otherwise infect corresponding bacteria without such phage resistance. In some such embodiments, bacteriophages against which therapeutic bacteria exhibit resistance are associated with dysbiosis and/or microbiome-dysfunction-associated diseases, disorders, or conditions.

In some embodiments, bacteriophage-resistant bacteria may be isolated from a biological tissue or fluid sample of a subject (e.g., a mammalian subject). For example, in some embodiments, bacteriophage-resistant bacteria may be isolated from bodily excretions of a mammalian subject, including, e.g., but not limited to saliva, mucus, urine, and/or stool (e.g., fecal sample). In some embodiments, bacteriophage-resistant bacteria may be genetically engineered in vitro or ex vivo. In some embodiments, bacteriophage-resistant bacteria may be generated in vivo, for example, by administering to a subject a composition comprising a nucleic acid sequence, wherein the nucleic acid sequence is delivered to host bacteria that are susceptible to such bacteriophages for genetic manipulation to become resistant to such bacteriophages.

### 1. CRISPR Systems (e.g., CRISPR-Cas Systems)

In some embodiments, therapeutic bacteria for use in compositions and/or methods described herein each comprise a clustered regularly interspaced short palindromic repeats (CRISPR) system comprising a CRISPR spacer that targets one or more bacteriophage sequences and a CRISPR-associated (Cas) polypeptide. In some such embodiments, a CRISPR system is characterized by sufficient flexibility that, when a therapeutic bacterium is infected by a bacteriophage which contains a targeted spacer sequence (protospacer), or a related variant of such a targeted spacer sequence (e.g., in some embodiments, a mutant bacteriophage with a protospacer that evolves at least one or more, e.g., at least two, at least three, at least four or more, mutations from a parent or target bacteriophage; or in some embodiments, a mutant bacteriophage with a protospacer that evolves 1-4 mutations from a parent or target bacteriophages), the therapeutic bacterium shows increased resistance to the infection relative to that observed for an otherwise comparable bacterium not comprising a CRISPR spacer that targets the bacteriophage or a related variant thereof. In some embodiments, such a therapeutic bacterium can show increased resistance to bacteriophage infection by at least 30% or more (including, e.g., at least 40%, at least 50%, at least 60%, at least 70%, at least 80%, at least 90%, at least 95%, or more), relative to that observed for an otherwise comparable bacterium not comprising a CRISPR spacer that targets the bacteriophage or a related variant thereof. In some such embodiments, bacteriophage resistance can be characterized by one or more methods as described in section entitled “Exemplary methods for characterization of bacteriophage resistance” below.

CRISPR-Cas systems evolved in bacteria to provide adaptive immunity against foreign genetic elements, including phages. CRISPRs are typically short partially palindromic sequences of 24-65 bp containing inner and terminal inverted repeats. Although isolated elements have been detected, they are generally arranged in clusters (up to about 20 or more per genome) of repeated units spaced by unique intervening 20-58 bp sequences.

CRISPR systems are found in approximately 40% and 90% of sequenced bacterial and archaeal genomes, respectively. A diverse array of CRISPR systems are also identified in non-pathogenic and commensal bacteria. Rho, M., et al. PLoS Genet. 8, e1002441 (2012); Soto-Perez, P. et al. Cell Host Microbe 26, 325-335.e5 (2019). CRISPR systems present in non-pathogenic and commensal bacteria that may be manipulated in accordance with the present disclosure include, for example, Type I-A, Type I-B, Type I-C, Type I-D, Type I-E, Type I-F, Type III-A, Type III-B, Type II-A, or Type II-B. In some embodiments, a CRISPR system present in non-pathogenic and commensal bacteria that may be manipulated in accordance with the present disclosure is Type I (e.g., Type I-C) or Type III (e.g., Type III-A, Type III-B). Various computer software and web resources are available for the analysis of and identification of CRISPR systems and CRISPR arrays that may be useful in the compositions and/or methods described herein. These tools include, but are not limited to, software for CRISPR detection, such as PILERCR, CRISPR Recognition Tool and CRISPRFinder; online repositories of pre-analyzed CRISPRs, such as CRISPRdb; and tools for browsing CRISPRs in microbial genomes, such as Pygram. As will be understood by a skilled artisan, CRISPR arrays from any one of such CRISPR systems may be used in accordance with the present disclosure. In some embodiments, therapeutic bacteria described herein may employ endogenous CRISPR arrays for modulating cell resistance to target bacteriophages. In some embodiments, therapeutic bacteria described herein may comprise a heterologous CRISPR array, including at least one Cas protein (e.g., ones described herein), at least one CRISPR spacer (e.g., ones described herein) and at least two CRISPR repeats (e.g., ones described herein) introduced therein for modulating cell resistance to target bacteriophages.

CRISPR spacers: Therapeutic bacteria used in accordance with the present disclosure each comprises one or more CRISPR spacers that targets one or more bacteriophage sequences, e.g., one or more characteristic sequence elements of bacteriophage(s). In some embodiments, such a CRISPR spacer can target a characteristic nucleic acid sequence element or transcription product thereof of one or more bacteriophages. Such CRISPR spacers can be naturally occurring or endogenously expressed in bacterial cells or introduced into such a cell by methods known in the art.

In some embodiments, a therapeutic bacterium described herein comprises at least one or more, including, e.g., at least two, at least three, at least four, at least five, or more, CRISPR spacer(s), each of which targets a different characteristic sequence element (e.g., a characteristic nucleic acid sequence element or transcription product thereof) of the same target bacteriophage. In some embodiments, a therapeutic bacterium described herein comprises at least one or more, including, e.g., at least two, at least three, at least four, at least five, or more, CRISPR spacer(s), each of which targets a characteristic sequence element (e.g., a characteristic nucleic acid sequence element or transcription product thereof) of a distinct target bacteriophage.

A CRISPR spacer typically is or comprises a sequence that is complementary to a characteristic sequence element (e.g., a characteristic nucleic acid sequence element or transcription product thereof or a protospacer) of a target bacteriophage such that it is able to bind to a characteristic sequence element of one or more bacteriophages or related variants thereof, resulting in cleavages of such a characteristic sequence element in the presence of an appropriate Cas polypeptide. In some embodiments, a CRISPR spacer is or comprises a sequence that is complementary (e.g., 100% complementary) to a characteristic sequence element (e.g., a characteristic nucleic acid sequence element or transcription product thereof or a protospacer) of a reference bacteriophage. In some embodiments, a CRISPR spacer is or comprises a sequence that has at least one base pair mismatch (including, e.g., at least two base pair mismatches, at least three base pair mismatches, at least four base pair mismatches, or more) to a characteristic sequence element (e.g., a characteristic nucleic acid sequence element or transcription product thereof or a protospacer) of a reference bacteriophage. In some embodiments, a CRISPR spacer is or comprises a sequence that has 1-4 base pair mismatch(es) to a characteristic sequence element (e.g., a characteristic nucleic acid sequence element or transcription product thereof or a protospacer) of a reference bacteriophage.

In some embodiments, a CRISPR spacer is or comprises a sequence that exists as a CRISPR spacer within a CRISPR locus of non-pathogenic and commensal bacteria found in a human gut, wherein such a CRISPR spacer matches a characteristic sequence element of a bacteriophage typically found in a human gut. For example, in some embodiments, a CRISPR spacer is or comprises a sequence that exists as a CRISPR spacer within a CRISPR locus of Eggerthella lenta, a human gut Actinobacteirum, for example, as described in Soto-Perez et al. Cell Host & Microbes (2019) 26: 1-11, the contents of which are incorporated herein by reference in their entirety.

In some embodiments, a CRISPR spacer is or comprises a sequence that is determined by computational approaches. For example, as described in Example 2, a CRISPR spacer according to some embodiments can be determined by matching portions of bacteriophage sequences to known CRISPR spacer sequences identified from bacterial hosts, e.g., gut bacteria.

Cas polypeptides or genes encoding the same: CRISPR structures or arrays are typically found in the vicinity of CRISPR-associated (Cas) genes. A variety of Cas genes or polypeptide(s) that are known in the art can be used in the compositions and/or methods described herein and the choice of Cas polypeptide(s) may vary with bacteriophages to be targeted by compositions and/or methods described herein. In some embodiments, a Cas protein can be selected based on its efficacy in conferring resistance to bacteriophage populations. Examples of Cas polypeptide(s) that may be useful in compositions and/or methods described herein include, but are not limited to Cas 1, Cas2, Cas3, Cas4, Cas5, Cash, Cas7, Cas8, Cas9, Cas10, and combinations thereof. In some embodiments, therapeutic bacteria may comprise a type III Cas polypeptide, e.g., a Cas10 polypeptide. In some embodiments, therapeutic bacteria may comprise a Cas RNA nuclease. In some embodiments, therapeutic bacteria described herein may employ endogenous Cas polypeptide(s). In some embodiments, therapeutic bacteria described herein may comprise a heterologous Cas polypeptide.

In some embodiments, a therapeutic bacterium described herein may comprise one or more Cas genes or polypeptides that are endogenous to such a therapeutic bacterium and one or more heterologous CRISPR spacers operatively associated with such one or more Cas genes or polypeptides.

In some embodiments, a therapeutic bacterium described herein may comprise one or more Cas genes or polypeptides that are heterologous to such a therapeutic bacterium and one or more CRISPR spacers that may be homologous or heterologous to such a therapeutic bacterium. In some such embodiments, one or more CRISPR spacers can be operatively associated with such one or more Cas genes or polypeptides.

Methods to modulate CRISPR-mediated immunity in cells are known in the art, for example, as described in U.S. Pat. No. 9,879,269 and U.S. 2016/0348120, the contents of each of which are incorporated herein by reference in their entireties for the purposes described herein. A skilled artisan, reading the present disclosure, will recognize that such methods and other methods known in the art can be used to generate therapeutic bacteria according to some embodiments described herein.

### 2. Mutants of Bacteriophage Receptors

In some embodiments, therapeutic bacteria for use in compositions and/or methods described herein each comprise at least one or more mutants of bacteriophage receptor(s) on bacterial cell surface. In some embodiments, one or more mutations in a bacteriophage receptor that confer resistance can be identified by exposing bacterial populations to phages, and selecting those bacteria that show improved survival. This strategy allows isolating some phage-resistant bacteria that have mutations of bacteriophage receptor(s) on the bacterial cell surface. These mutations can then be engineered into desired bacterial strains to confer resistance. In some such embodiments, therapeutic bacteria are each genetically engineered to express at least one or more mutants of bacteriophage receptor(s) on bacterial cell surface. In some such embodiments, therapeutic bacteria are isolated or purified from a biological sample from a subject. In some embodiments, such therapeutic bacteria may be isolated or purified from a fecal sample from an individual who is determined to be less susceptible to inflammatory bowel disease (e.g., a healthy individual).

### D. Engineered Bacterial Populations

The present disclosure, among other things, provides engineered populations of therapeutic bacteria (e.g., ones as described herein) that are resistant one or more bacteriophages. Such populations of therapeutic bacteria can be useful for treatment of microbiome-dysfunction-associated diseases or disorders.

In some embodiments, an engineered population of therapeutic bacteria is or comprises an enriched or purified population of therapeutic bacteria (e.g., ones as described herein). For example, in some embodiments, naturally occurring phage-resistant bacterial cells are enriched or purified from mixed populations of bacteria. In some embodiments, such naturally occurring phage-resistant bacterial cells may comprise CRISPR spacers that target one or more bacteriophages. In some embodiments, such naturally occurring phage-resistant cells may also have mutations affecting cell wall properties that inhibit phage infection. In some embodiments, such naturally occurring phage-resistant bacterial cells may be isolated or purified from biological samples of a human subject or a population of human subjects. In some embodiments, such isolated or purified phage-resistant bacterial cells may be cultured in vitro for clonal selection and/or cell expansion.

In some embodiments, phage-resistant bacterial cells may be isolated by exposing susceptible bacterial cultures to a target phage. Typically, the majority of the bacterial cells (>90%) may be eliminated in the presence of a target phage (e.g., after 24 hours or longer, including, e.g., 48 hours, 72 hours, or longer). Bacterial cells that survive may be considered as phage-resistant candidates. In some embodiments, such surviving bacterial cells (after the first exposure to a target phage) may be subjected to at least a second phage exposure, e.g., exposure to the same target or a different phage, and bacterial cells that survive after such the second or more phage exposure can be characterized as phage-resistant. Such phage-resistant cells may be cultured in vitro for clonal selection and/or cell expansion.

In some embodiments, isolated phage-resistant bacterial cells may be sequenced to identify mutation(s) or CRISPR spacers that confer resistance. Such information can be useful for genetically engineering phage-resistant bacteria in some embodiments. Accordingly, in some embodiments, an engineered population of therapeutic bacteria is or comprises a population of therapeutic bacteria (e.g., ones as described herein) comprising non-pathogenic and commensal bacteria that are genetically engineered to be resistant to one or more target bacteriophages, e.g., as described in section entitled “Exemplary bacteriophage-resistant bacteria” described above.

In some embodiments, therapeutic bacteria (e.g., ones described herein) in a population are all from a single strain. In some embodiments, therapeutic bacteria (e.g., ones described herein) in a population are all from a single clone. In some embodiments, therapeutic bacteria (e.g., ones described herein) in a population includes a collection of bacterial strains. In some embodiments, therapeutic bacteria (e.g., ones described herein) in a population include at least one or more strain that is found in human microbiome. In some embodiments, therapeutic bacteria (e.g., ones described herein) in a population include a genetically engineered variant. In some embodiments, therapeutic bacteria (e.g., ones described herein) in a population comprises a plurality of bacterial strains, each of which is present in human microbiome; in some such embodiments, provided such a population includes individual strains in different relative amounts (e.g., to one another and/or to a reference strain) than found in human population (on average, and/or in particular sub-population, and/or in particular human or collection thereof).

In some embodiments, therapeutic bacteria (e.g., ones described herein) in a population are of the same bacterial species, wherein at least two or more (e.g., at least three, at least four, at least five, or more) subsets of such therapeutic bacteria comprise one or more different genetic modifications (e.g., a distinct CRISPR spacer and/or a distinct bacteriophage receptor mutant) that confer resistance to one or more bacteriophages.

In some embodiments, therapeutic bacteria (e.g., ones described herein) in a population are of different bacterial genera and/or species, wherein each subset of distinct bacterial genera and/or species comprises one or more different genetic modifications (e.g., a distinct CRISPR spacer and/or a distinct bacteriophage receptor mutant) that confer resistance to one or more bacteriophages.

### E. Exemplary Methods for Characterization of Bacteriophage Resistance

Bacterial cells (e.g., whether isolated from a biological sample or upon genetic manipulation) can be assessed or characterized for their bacteriophage resistance to identify and select phage-resistant bacteria.

In some embodiments, populations (e.g., clonal populations) of bacteria that are speculated or expected to have developed resistance to bacteriophage as a result of isolation and/or engineering can be cultivated in appropriate liquid media, and challenged with a single characterised/uncharacterised bacteriophage, cocktails of multiple characterised/uncharacterised bacteriophage, or biological compositions likely to comprise characterised/uncharacterised bacteriophage, which have been shown to infect the clonal bacterial population prior to experimental intervention. Bacteriophage-challenged bacterial cultures with growth statistically similar to bacterial cultures not challenged with bacteriophage can be considered to have developed complete resistance to the challenging bacteriophage populations. Bacteriophage-challenged bacteriophage-resistant cultures may also have statistically greater growth rates than bacteriophage-susceptible cultures and these would be characterized as partially resistant. This method can be adapted where clonal and non-clonal population of bacteria can be challenged with bacteriophage prior to cultivation, or whilst in lag phase.

Additionally or alternatively, bacteriophage resistant phenotypes and/or genotypes of bacteria can be identified using solid media through plaque assays. For example, bacteria that are speculated or expected to have developed resistance to bacteriophage as a result of experimental intervention (e.g., isolation and/or engineering) are grown on solid media and challenged with serial dilution of appropriate buffers comprising a single characterised/uncharacterised bacteriophage, cocktails of multiple characterised/uncharacterised bacteriophage, or biological compositions likely to comprise characterised/uncharacterised bacteriophage, which have been shown to infect the clonal bacterial population prior to experimental intervention. Bacterial colonies surviving bacteriophage challenge can be considered bacteriophage resistant when controls comprising bacteria prior to experimental intervention universally develop plaques.

In some embodiments, underlying genetic mechanisms of bacterial resistance to bacteriophage populations can be determined using comparative genomics. For example, in some embodiments, genomes of bacterial isolates showing bacteriophage resistance are compared to bacteriophage sensitive populations of the same bacterial strain.

### II. Exemplary Compositions

The present disclosure, among other things, also provides compositions that exhibit bacterial resistance to bacteriophages, e.g., bacteriophages associated with microbiome-dysfunction-associated diseases or disorders or dysbiosis. In some embodiments, such compositions can be more useful in treating microbiome-associated diseases or disorders than certain prior technologies including, for example, administration of conventional probiotics and/or beneficial bacteria, which can still be infected and depleted by bacteriophages present in the microbiome of subjects to be treated.

### A. Pharmaceutical Compositions Comprising Therapeutic Bacteria

One aspect described herein relates to pharmaceutical or therapeutic compositions comprising an engineered population of therapeutic bacteria (e.g., ones as described herein) that (i) are non-pathogenic and commensal in a subject to be administered; and (ii) are resistant to one or more target bacteriophages. In some embodiments, a pharmaceutical or therapeutic composition comprises an engineered bacterial population as described in section entitled “Engineered bacterial populations” above.

In some embodiments, therapeutic bacteria included in a pharmaceutical or therapeutic composition described herein comprises at least one or more (including, e.g., at least two, at least three, at least four, at least five, or more) isolated, purified, or cultured bacteria selected from the group consisting of Bacillus, Bacteroides, Bifidobacterium, Coprococcus, Clostridium, Collinsella, Desulfomonas, Dorea, Escherichia, Eubacterium, Fusobacterium, Gemmiger, Lactobacillus, Lactoccucs, Monilia, Peptostreptococcus, Propionibacterium, Ruminococcus, and combinations thereof. In some embodiments, therapeutic bacteria included in a therapeutic composition described herein comprises Bacteroides, Bifidobacterium, Clostridium, Escherichia, Lactobacillus, Lactoccucs, or combinations thereof. In some embodiments, such bacteria may be autologous. In some embodiments, such bacteria may be allogeneic.

### B. Pharmaceutical Compositions Comprising Nucleic Acids for In Vivo Genetic Manipulation of Host Bacteria to Become Phage-Resistant

While pharmaceutical compositions comprising exogenous therapeutic bacteria (e.g., ones as described herein) can be administered to subjects who are in need thereof, in some embodiments, such subjects' endogenous bacteria of microbiota that are susceptible to one or more target bacteriophages can be genetically engineered in vivo to become resistant to such target bacteriophages, for example, by administering a pharmaceutical composition comprising a nucleic acid sequence for modulating resistance of such host bacteria. Accordingly, another aspect provided herein relates to a pharmaceutical composition comprising a nucleic acid sequence for altering genetic information of host non-pathogenic and commensal bacteria in a subject in need thereof so that such host bacteria are genetically engineered to become resistant to target bacteriophages.

In some embodiments, a nucleic acid sequence for altering genetic information of host commensal bacteria comprises one or more CRISPR spacers that target one or more target bacteriophages. In some embodiments, a nucleic acid sequence for altering genetic information of host commensal bacteria comprises one or more nucleotide sequences encoding one or more Cas polypeptides (e.g., ones described herein).

In some embodiments, a nucleic acid sequence for altering genetic information of host commensal bacteria comprises one or more nucleotide sequences encoding one or more mutants of bacteriophage receptors on bacterial cell surface.

Methods for delivering a composition comprising a nucleic acid sequence are known in the art; one skilled in the art will understand that, in some embodiments, such a nucleic acid sequence may be delivered by a recombinant bacteriophage (e.g., as a carrier), while in some embodiments, such a nucleic acid sequence may be delivered by a vector, cosmid, phagemid or transposon.

In some embodiments, a nucleic acid sequence in accordance with the present disclosure may be delivered by an expression vector. Expression vectors that may be useful for delivering a nucleic acid sequence for modulating a bacterial cell's resistance to bacteriophage include, but are not limited to, viral vectors based on vaccinia virus, poliovirus, adenovirus, adeno-associated virus, SV40, herpes simplex virus, human immunodeficiency virus, retrovirus (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus) and other recombinant vectors.

In some examples, a vector can comprise one or more transcription and/or translation control elements. Depending on the host/vector system utilized, any of a number of suitable transcription and translation control elements, including constitutive and inducible promoters, transcription enhancer elements, transcription terminators, etc. can be used in the expression vector.

In some embodiments, a vector can be autonomously replicated in a host cell (episomal vector), or may be integrated into the genome of a host cell, and replicated along with the host genome (non-episomal mammalian vector). Integrating vectors typically contain at least one sequence homologous to the bacterial chromosome that allows for recombination to occur between homologous DNA in the vector and the bacterial chromosome. Integrating vectors may also comprise bacteriophage or transposon sequences. Episomal vectors, or plasmids are circular double-stranded DNA loops into which additional DNA segments can be ligated. In some embodiments, plasmids capable of stable maintenance in a host are used as expression vectors when using recombinant DNA techniques.

Regulatory sequences include those that direct constitutive expression of a nucleotide sequence as well as those that direct inducible expression of the nucleotide sequence only under certain environmental conditions. A bacterial promoter is any DNA sequence capable of binding bacterial RNA polymerase and initiating the downstream (3′) transcription of a coding sequence (e.g., structural gene) into mRNA. A promoter will have a transcription initiation region, which is usually placed proximal to the 5′ end of the coding sequence. This transcription initiation region typically includes an RNA polymerase binding site and a transcription initiation site. A bacterial promoter may also have a second domain called an operator, which may overlap an adjacent RNA polymerase binding site at which RNA synthesis begins. The operator permits negative regulated (inducible) transcription, as a gene repressor protein may bind the operator and thereby inhibit transcription of a specific gene. Constitutive expression may occur in the absence of negative regulatory elements, such as the operator. In addition, positive regulation may be achieved by a gene activator protein binding sequence, which, if present is usually proximal (5′) to the RNA polymerase binding sequence.

In some embodiments, a nucleic acid sequence in accordance with the present disclosure may be delivered by a recombinant phage. In some such embodiments, a recombinant phage can be a phagemid particle, e.g., a bacteriophage-derived particle that comprises a phagemid comprising a nucleic acid sequence (e.g., as described herein) for modulating a bacterial cell's resistance to a target bacteriophage but does not contain a bacteriophage genome. For example, in some embodiments, a phagemid may comprise a nucleic acid sequence encoding a CRISPR spacer (e.g., ones as described herein). Additionally or alternatively, a phagemid may comprise a nucleic acid sequence encoding a relevant Cas polypeptide.

Pharmaceutical compositions provided herein can include those suitable for oral including buccal and sub-lingual, intranasal, topical, transdermal, transdermal patch, pulmonary, vaginal, rectal, suppository, mucosal, systemic, or parenteral including intramuscular, intraarterial, intrathecal, intradermal, intraperitoneal, subcutaneous, and intravenous administration or in a form suitable for administration by aerosolization, inhalation or insufflation.

In some embodiments, a pharmaceutical composition described herein can include carriers and excipients (including but not limited to buffers, carbohydrates, lipids, mannitol, proteins, polypeptides or amino acids such as glycine, antioxidants, bacteriostats, chelating agents, suspending agents, thickening agents and/or preservatives), metals (e.g., iron, calcium), salts, vitamins, minerals, water, oils including those of petroleum, animal, vegetable or synthetic origin, such as peanut oil, soybean oil, mineral oil, sesame oil and the like, saline solutions, aqueous dextrose and glycerol solutions, flavoring agents, coloring agents, detackifiers and other acceptable additives, adjuvants, or binders, other pharmaceutically acceptable auxiliary substances as required to approximate physiological conditions, such as pH buffering agents, tonicity adjusting agents, emulsifying agents, wetting agents and the like. Examples of excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

Non-limiting examples of pharmaceutically-acceptable excipients suitable for use in accordance with the present disclosure include granulating agents, binding agents, lubricating agents, disintegrating agents, sweetening agents, glidants, anti-adherents, anti-static agents, surfactants, antioxidants, gums, coating agents, coloring agents, flavouring agents, dispersion enhancer, disintegrant, coating agents, plasticizers, preservatives, suspending agents, emulsifying agents, plant cellulosic material and spheronization agents, and any combination thereof.

In some embodiments, a pharmaceutical composition described herein can be substantially free of preservatives. In some applications, the composition may contain at least one preservative.

In some embodiments, a pharmaceutical composition described herein can be encapsulated within a suitable vehicle, for example, a liposome, a microspheres, or a microparticle. Microspheres formed of polymers or proteins can be tailored for passage through the gastrointestinal tract directly into the blood stream. Alternatively, the compound can be incorporated and the microspheres, or composite of microspheres, and implanted for slow release over a period of time ranging from days to months.

In some embodiments, a pharmaceutical composition described herein can be formulated as a sterile solution or suspension. Such a pharmaceutical composition can be sterilized by conventional techniques or may be sterile filtered. The resulting aqueous solutions may be packaged for use as is, or lyophilized. The lyophilized preparation of therapeutic bacteria (e.g., ones described herein) can be packaged in a suitable form for oral administration, for example, capsule or pill.

In some embodiments, a pharmaceutical composition described herein can be administered topically and can be formulated into a variety of topically administrable compositions, such as solutions, suspensions, lotions, gels, pastes, medicated sticks, balms, creams, and ointments. Such pharmaceutical compositions can contain solubilizers, stabilizers, tonicity enhancing agents, buffers and preservatives.

In some embodiments, a pharmaceutical composition described herein can be formulated in a rectal composition such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, PEG, and the like. In suppository forms of the compositions, a low-melting wax such as a mixture of fatty acid glycerides, optionally in combination with cocoa butter, can be used.

In practicing the methods of treatment or use provided herein, therapeutically-effective amounts of microbial compositions (e.g., therapeutic bacteria) and/or pharmaceutical compositions described herein are administered to a subject (e.g., a human subject) having a microbiome-dysfunction-associated disease, disorder, or condition to be treated. A therapeutically-effective amount can vary widely depending on the severity of the disease, the age and relative health of the subject, potency of the formulation, and other factors. Subjects can be, for example, humans, elderly adults, adults, adolescents, pre-adolescents, children, toddlers, infants, or neonates. A subject can be a patient. A subject can be an individual enrolled in a clinical study. A subject can be a laboratory animal, for example, a mammal, or a rodent.

Pharmaceutical compositions can be formulated using one or more physiologically-acceptable carriers comprising excipients and auxiliaries, which facilitate processing of the microorganisms into preparations that can be used pharmaceutically. Formulation can be modified depending upon the route of administration chosen. Pharmaceutical compositions described herein can be manufactured in a conventional manner, for example, by means of conventional mixing, dissolving, granulating, vitrification, spray-drying, lyophilizing, dragee-making, levigating, encapsulating, entrapping, emulsifying or compression processes.

In some embodiments, a pharmaceutical composition is manufactured in a dry form, for example, by spray-drying or lyophilization. In some embodiments, a pharmaceutical composition is formulated as a liquid capsule to maintain the liquid form of therapeutic bacteria (e.g., ones described herein).

### C. Other Compositions and Formulations

Compositions described herein can be formulated for various applications involving microbiome. In some embodiments, compositions described herein can be formulated as pharmaceutical or therapeutic compositions as described above. In some embodiments, compositions described herein can be included in cosmetics compositions. In some embodiments, compositions described herein can be included in food or beverage products. In some embodiments, compositions described herein can be included in nutritional supplements.

In some embodiments, compositions comprising therapeutic bacteria (e.g., ones described herein) can be formulated as a nutritional or dietary supplement. For example, in some embodiments, therapeutic bacteria (e.g., ones described herein) can be incorporated with vitamin supplements. In some embodiments, compositions comprising therapeutic bacteria (e.g., ones as described herein) can be formulated in a chewable form such as a probiotic gummy.

In some embodiments, compositions comprising therapeutic bacteria (e.g., ones as described herein) can be incorporated into a form of food and/or drink. Non-limiting examples of food and drinks in which therapeutic bacteria can be incorporated include, for example, bars, shakes, juices, infant formula, beverages, frozen food products, fermented food products, and cultured dairy products such as yogurt, yogurt drink, cheese, acidophilus drinks, and kefir.

In some embodiments, compositions comprising therapeutic bacteria (e.g., ones as described herein) can be formulated for use in cosmetics (e.g., skincare products or make-up products). One or more therapeutic bacteria described herein can be used to create a cosmetic formulation comprising an effective amount of therapeutic bacteria (e.g., ones described herein) for treating a subject suffering from or susceptible to a skin disorder involving microbiome. In some embodiments, compositions comprising therapeutic bacteria (e.g., ones described herein) can be included in lotions, creams, moisturizers, powder, etc.

In some embodiments, compositions described herein can be administered by a suitable method for delivery to any part of the gastrointestinal tract of a subject including oral cavity, mouth, esophagus, stomach, duodenum, small intestine regions including duodenum, jejunum, ileum, and large intestine regions including cecum, colon, rectum, and anal canal. In some embodiments, compositions described herein may be formulated for delivery to the ileum and/or colon regions of the gastrointestinal tract.

In some embodiments, compositions described herein may be administered orally, for example, through a capsule, pill, powder, tablet, gel, or liquid, designed to release such compositions in a gastrointestinal tract. In some embodiments, compositions described herein may be administered by injection, for example, for a formulation comprising butyrate, propionate, acetate, and/or short-chain fatty acids. In some embodiments, compositions described herein may be applied to skin, for example, in the form of cream, liquid, or patch. In some embodiments, compositions described herein may be administered in a form of a suppository and/or enema. In some embodiments, a combination of administration routes may be utilized.

In some embodiments, compositions described herein can be administered as part of a fecal transplant process. For example, in some embodiments, such a composition can be administered to a subject by a tube, for example, nasogastric tube, nasojejunal tube, nasoduodenal tube, oral gastric tube, oral jejunal tube, or oral duodenal tube. In some embodiments, a composition can be administered to a subject by colonoscopy, endoscopy, sigmoidoscopy, and/or enema.

In some embodiments, a bacterial composition comprising therapeutic bacteria (e.g., ones described herein) is formulated such that the one or more therapeutic bacteria can replicate once they are delivered to a target habitat (e.g. the gut). In one non-limiting example, such a bacterial composition is formulated in a pill, such that the pill has a shelf life of at least 1, 2, 3, 4, 5, 6, 7, 8, 9, 10, 11, or 12 months. In another non-limiting example, the storage of a bacterial composition is formulated so that therapeutic bacteria included therein can reproduce once they are in the gut. In some embodiments, other components may be added to aid in the shelf life of such a bacterial composition. In some embodiments, one or more therapeutic bacteria may be formulated in a manner that it is able to survive in a non-natural environment. For example, a bacterium that is native to the gut may not survive in an oxygen-rich environment. To overcome this limitation, such a bacterium may be formulated in a pill that can reduce or eliminate the exposure to oxygen. Other strategies to enhance the shelf-life of therapeutic bacteria may include other microbes (e.g. if the bacterial consortia comprises a composition whereby one or more strains is helpful for the survival of one or more strains).

In some embodiments, a bacterial composition comprising therapeutic bacteria (e.g., ones described herein) is lyophilized (e.g., freeze-dried) and formulated as a powder, tablet, enteric-coated capsule (e.g. for delivery to ileum/colon), or pill that can be administered to a subject by any suitable route. Such a lyophilized formulation can be mixed with a saline or other solution prior to administration.

In some embodiments, a bacterial composition comprising therapeutic bacteria (e.g., ones described herein) is formulated for oral administration, for example, as an enteric-coated capsule or pill, for delivery of the contents of such a formulation to the ileum and/or colon regions of a subject.

In some embodiments, a bacterial composition comprising therapeutic bacteria (e.g., ones described herein) is formulated for oral administration. In some embodiments, a bacterial composition comprising therapeutic bacteria (e.g., ones described herein) is formulated as an enteric-coated pill or capsule for oral administration. In some embodiments, a bacterial composition comprising therapeutic bacteria (e.g., ones described herein) is formulated for delivery of such therapeutic bacteria to the ileum region of a subject. In some embodiments, a bacterial composition comprising therapeutic bacteria (e.g., ones described herein) is formulated for delivery of such therapeutic bacteria to the colon region (e.g. upper colon) of a subject. In some embodiments, a bacterial composition comprising therapeutic bacteria (e.g., ones described herein) is formulated for delivery of such therapeutic bacteria to the ileum and colon regions of a subject.

In some embodiments, an enteric-coating can be used to protect the contents of an oral formulation, for example, pill or capsule, from the acidity of the stomach and provide delivery to the ileum and/or upper colon regions. Non-limiting examples of enteric coatings include pH sensitive polymers (e.g., eudragit FS30D), methyl acrylate-methacrylic acid copolymers, cellulose acetate succinate, hydroxy propyl methyl cellulose phthalate, hydroxy propyl methyl cellulose acetate succinate (e.g., hypromellose acetate succinate), polyvinyl acetate phthalate (PVAP), methyl methacrylate-methacrylic acid copolymers, shellac, cellulose acetate trimellitate, sodium alginate, zein, other polymers, fatty acids, waxes, shellac, plastics, and plant fibers. In some embodiments, an enteric coating is formed by a pH sensitive polymer. In some embodiments, an enteric coating is formed by eudragit FS30D.

In some embodiments, an enteric coating can be designed to dissolve at any suitable pH. In some embodiments, an enteric coating is designed to dissolve at a pH greater than about pH 6.5 to about pH 7.0. In some embodiments, an enteric coating is designed to dissolve at a pH greater than about pH 6.5. In some embodiments, an enteric coating is designed to dissolve at a pH greater than about pH 7.0. In some embodiments, an enteric coating can be designed to dissolve at a pH greater than about: 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, or 7.5 pH units.

In some embodiments, compositions described herein (e.g., pharmaceutical compositions) are formulated for delivery of a bacterial population (e.g., ones described herein) to the colon. Examples of such formulations include, but are not limited to, pH sensitive compositions, more specifically, buffered sachet formulations or enteric polymers that release their contents when the pH becomes alkaline after the enteric polymers pass through the stomach. When a pH sensitive composition is used for formulating a pharmaceutical preparation, the pH sensitive composition is preferably a polymer whose pH threshold of the decomposition of the composition is between about 6.8 and about 7.5. Such a numeric value range is a range in which the pH shifts toward the alkaline side at a distal portion of the stomach, and hence is a suitable range for use in the delivery to the colon.

Another embodiment of a preparation (e.g., a pharmaceutical preparation) useful for delivery of bacterial composition to the colon is one that ensures the delivery to the colon by delaying the release of the contents (e.g., the bacterial composition) by approximately 3 to 5 hours, which corresponds to the small intestinal transit time. In one embodiment of a pharmaceutical preparation for delayed release, a hydrogel is used as a shell. The hydrogel is hydrated and swells upon contact with gastrointestinal fluid, with the result that the contents are effectively released (released predominantly in the colon). Delayed release dosage units include drug-containing compositions having a material which coats or selectively coats a drug or active ingredient to be administered. Examples of such a selective coating material include in vivo degradable polymers, gradually hydrolyzable polymers, gradually water-soluble polymers, and/or enzyme degradable polymers. A wide variety of coating materials for efficiently delaying the release is available and includes, for example, cellulose-based polymers such as hydroxypropyl cellulose, acrylic acid polymers and copolymers such as methacrylic acid polymers and copolymers, and vinyl polymers and copolymers such as polyvinylpyrrolidone.

Examples of a composition enabling the delivery to the colon further include bioadhesive compositions which specifically adhere to the colonic mucosal membrane (for example, a polymer described in the specification of U.S. Pat. No. 6,368,586) and compositions into which a protease inhibitor is incorporated for protecting particularly a biopharmaceutical preparation in the gastrointestinal tracts from decomposition due to an activity of a protease.

Another example of a system enabling the delivery to the colon is a system of delivering a composition to the colon by pressure change in such a way that the contents are released by utilizing pressure change caused by generation of gas in bacterial fermentation at a distal portion of the stomach. Such a system is not particularly limited, and a more specific example thereof is a capsule which has contents dispersed in a suppository base and which is coated with a hydrophobic polymer (for example, ethyl cellulose).

Another example of the system enabling the delivery to the colon is a system of delivering a composition to the colon, the system being specifically decomposed by an enzyme (for example, a carbohydrate hydrolase or a carbohydrate reductase) present in the colon. Such a system is not particularly limited, and more specific examples thereof include systems which use food components such as non-starch polysaccharides, amylose, xanthan gum, and azopolymers.

In some embodiments, therapeutic bacteria (e.g., ones described herein) are formulated as a population of spores. Spore-containing formulations can be administered by any suitable route described herein. Orally administered spore-containing formulations can survive the low pH environment of the stomach. The amount of spores employed can be, for example, from about 1% w/w to about 99% w/w of the entire formulation.

In some embodiments, a formulation comprises one or more recombinant bacteria or bacteria that have been genetically modified. In other embodiments, one or more bacteria are not modified or recombinant. In some embodiments, a formulation comprises bacteria that can be regulated, for example, bacteria comprising an operon or promoter to control bacterial growth. Bacteria can be produced, grown, or modified using any suitable methods, including recombinant methods.

A formulation can be customized for a subject. A custom formulation can comprise, for example, a prebiotic, a probiotic, an antibiotic, or a combination of active agents described herein. Data specific to the subject comprising for example age, gender, and weight can be combined with an analysis result to provide a therapeutic agent customized to the subject. For example, a subject's microbiome found to be high in a specific bacteriophage relative to a sub-population of healthy subjects matched for age and gender can be provided with a therapeutic and/or cosmetic formulation comprising an isolated or enriched population of phage-resistant bacteria that target the identified bacteriophage.

Compositions provided herein can be stored at any suitable temperature. Formulations can be stored in cold storage, for example, at a temperature of about −80° C., about −20° C., about −4° C., or about 4° C. In some embodiments, formulations can be prepared for storage temperature of about 0° C., about 1° C., about 2° C., about 3° C., about 4° C., about 5° C., about 6° C., about 7° C., about 8° C., about 9° C., about 10° C., about 12° C., about 14° C., about 16° C., about 20° C., about 22° C., or about 25° C. In some embodiments, the storage temperature is between about 2° C. to about 8° C. In some embodiments, storage of microbial compositions (e.g., comprising therapeutic bacteria described herein) at low temperatures, for example from about 2° C. to about 8° C., can keep the microbes alive and increase the efficiency of the composition, for example, when present in a liquid or gel formulation. In some embodiments, storage at freezing temperature, below 0° C., with a cryoprotectant can further extend stability.

The pH of a composition described herein can range from about 3 to about 12 depending on applications (e.g., delivery to a gut vs. delivery to skin). The pH of such a composition can be, for example, from about 3 to about 4, from about 4 to about 5, from about 5 to about 6, from about 6 to about 7, from about 7 to about 8, from about 8 to about 9, from about 9 to about 10, from about 10 to about 11, or from about 11 to about 12 pH units. The pH of the composition can be, for example, about 3, about 4, about 5, about 6, about 7, about 8, about 9, about 10, about 11, or about 12 pH units. The pH of the composition can be, for example, at least 3, at least 4, at least 5, at least 6, at least 7, at least 8, at least 9, at least 10, at least 11 or at least 12 pH units. The pH of the composition can be, for example, at most 3, at most 4, at most 5, at most 6, at most 7, at most 8, at most 9, at most 10, at most 11, or at most 12 pH units. If the pH is outside the range desired by the formulator, the pH can be adjusted by using sufficient pharmaceutically-acceptable acids and bases. In some embodiments, the pH of the composition is between about 4 and about 6.

### D. Optional Additives

In some embodiments, compositions described herein may comprise a prebiotic. In some embodiments, a prebiotic may be or comprise inulin. The inulin can serve as an energy source for the bacterial formulation.

In some embodiments, compositions described herein may comprise one or more active agents or therapeutic agents. Exemplary active agents or therapeutic agents may include, but are not limited to antibiotics, prebiotics, probiotics, glycans (e.g., as decoys that can limit specific bacterial/viral binding to the intestinal wall), bacteriophages, microorganisms and the like.

In some embodiments, compositions described herein can may comprise one or more agents for enhancing stability and/or survival of bacterial formulations. Non-limiting example of such stabilizing agents include genetic elements, glycerin, ascorbic acid, skim milk, lactose, tween, alginate, xanthan gum, carrageenan gum, mannitol, palm oil, poly-L-lysine (POPL), and combinations thereof.

### E. Exemplary Dosage Forms

The appropriate quantity of a pharmaceutical composition to be administered, the number of treatments, and unit dose can vary according to a subject and/or the disease state of the subject.

Pharmaceutical compositions described herein can be in unit dosage forms suitable for single administration of precise dosages. In unit dosage form, a pharmaceutical formulation described herein can be divided into unit doses containing appropriate quantities of one or more microbial compositions. The unit dosage can be in the form of a package containing discrete quantities of the formulation. Non-limiting examples are liquids in vials or ampoules. Aqueous suspension compositions can be packaged in single-dose non-reclosable containers. The composition can be in a multi-dose format. Multiple-dose reclosable containers can be used, for example, in combination with a preservative. Formulations for parenteral injection can be presented in unit dosage form, for example, in ampoules, or in multi-dose containers with a preservative.

In some embodiments, dosage can be in the form of a solid, semi-solid, or liquid composition. Non-limiting examples of dosage forms suitable for use in accordance with the present disclosure include feed, food, pellet, lozenge, liquid, elixir, aerosol, inhalant, spray, powder, tablet, pill, capsule, gel, geltab, nanosuspension, nanoparticle, microgel, suppository troches, aqueous or oily suspensions, ointment, patch, lotion, dentifrice, emulsion, creams, drops, dispersible powders or granules, emulsion in hard or soft gel capsules, syrups, phytoceuticals, nutraceuticals, dietary supplement, and any combination thereof.

Therapeutic bacteria (e.g., ones described herein) can be present in a suitable concentration in a pharmaceutical composition. The concentration of therapeutic bacteria can be for example, from about 10¹to about 10¹⁸ colony forming units (CFU). The concentration of therapeutic bacteria (e.g., ones described herein) can be, for example, at least 10¹, at least 10², at least 10³, at least 10⁴, at least 10⁵, at least 10⁶, at least 10⁷, at least 10⁸, at least 10⁹, at least 10¹⁰, at least 10¹¹, at least 10¹², at least 10¹³, at least 10¹⁴, at least 10¹⁵, at least 10¹⁶, at least 10¹⁷, or at least 10¹⁸CFU. The concentration of therapeutic bacteria (e.g., ones described herein) can be, for example, at most 10¹, at most 10², at most 10³, at most 10⁴, at most 10⁵, at most 10⁶, at most 10⁷, at most 10⁸, at most 10⁹, at most 10¹⁰, at most 10¹¹, at most 10¹², at most 10¹³, at most 10¹⁴, at most 10¹⁵, at most 10¹⁶, at most 10¹⁷, or at most 10¹⁸ CFU. In some embodiments, the concentration of therapeutic bacteria (e.g., ones described herein) is from about 10⁸ CFU to about 10⁹ CFU.

Pharmaceutical compositions described herein can be formulated with a suitable therapeutically-effective concentration of prebiotic. For example, a therapeutically-effective concentration of a prebiotic can be at least about 1 mg/ml, about 2 mg/ml, about 3 mg/ml, about 4 mg/ml, about 5 mg/ml, about 10 mg/ml, about 15 mg/ml, about 20 mg/ml, about 25 mg/ml, about 30 mg/ml, about 35 mg/ml, about 40 mg/ml, about 45 mg/ml, about 50 mg/ml, about 55 mg/ml, about 60 mg/ml, about 65 mg/ml, about 70 mg/ml, about 75 mg/ml, about 80 mg/ml, about 85 mg/ml, about 90 mg/ml, about 95 mg/ml, about 100 mg/ml, about 110 mg/ml, about 125 mg/ml, about 130 mg/ml, about 140 mg/ml, or about 150 mg/ml. For example, a therapeutically-effective concentration of a prebiotic can be at most about 1 mg/ml, about 2 mg/ml, about 3 mg/ml, about 4 mg/ml, about 5 mg/ml, about 10 mg/ml, about 15 mg/ml, about 20 mg/ml, about 25 mg/ml, about 30 mg/ml, about 35 mg/ml, about 40 mg/ml, about 45 mg/ml, about 50 mg/ml, about 55 mg/ml, about 60 mg/ml, about 65 mg/ml, about 70 mg/ml, about 75 mg/ml, about 80 mg/ml, about 85 mg/ml, about 90 mg/ml, about 95 mg/ml, about 100 mg/ml, about 110 mg/ml, about 125 mg/ml, about 130 mg/ml, about 140 mg/ml, or about 150 mg/ml. For example, a therapeutically-effective concentration of a prebiotic can be about 1 mg/ml, about 2 mg/ml, about 3 mg/ml, about 4 mg/ml, about 5 mg/ml, about 10 mg/ml, about 15 mg/ml, about 20 mg/ml, about 25 mg/ml, about 30 mg/ml, about 35 mg/ml, about 40 mg/ml, about 45 mg/ml, about 50 mg/ml, about 55 mg/ml, about 60 mg/ml, about 65 mg/ml, about 70 mg/ml, about 75 mg/ml, about 80 mg/ml, about 85 mg/ml, about 90 mg/ml, about 95 mg/ml, about 100 mg/ml, about 110 mg/ml, about 125 mg/ml, about 130 mg/ml, about 140 mg/ml, or about 150 mg/ml.

In some embodiments, pharmaceutical compositions described herein be administered, for example, 1, 2, 3, 4, 5, or more times daily. In some embodiments, pharmaceutical compositions described herein can be administered, for example, daily, every other day, three times a week, twice a week, once a week, or at other appropriate intervals for treatment of the condition.

### F. Kits

Compositions described herein can be packaged as a kit. In some embodiments, a kit includes written instructions on the administration/use of the composition. The written material can be, for example, a label. The written material can suggest conditions methods of administration. The instructions provide the subject and the supervising physician with the best guidance for achieving the optimal clinical outcome from the administration of the therapy. The written material can be a label. In some embodiments, the label can be approved by a regulatory agency, for example the U.S. Food and Drug Administration (FDA), the European Medicines Agency (EMA), or other regulatory agencies.

### III. Exemplary Uses

Technologies including therapeutic bacteria, compositions, and methods provided herein can be useful for treatment and/or prevention of a microbiome-dysfunction-associated disease, disorder, or condition. Accordingly, technologies provided herein are amenable to subjects suffering from or susceptible to a microbiome-dysfunction-associated disease, disorder, or condition. In some embodiments, technologies provided herein are amenable to subjects suffering from or susceptible to a gut microbiome-dysfunction-associated disease, disorder, or condition.

In some aspects, provided are methods comprising a step of exposing a subject suffering from or susceptible to a microbiome-dysfunction-associated disease, disorder, or condition, to a population of therapeutic bacteria (e.g., ones described herein) or a composition described herein (including, e.g., a pharmaceutical composition, a cosmetic composition, food or beverage, or a nutritional supplement).

In some embodiments, therapeutic bacteria exposed to a subject in need thereof (e.g., a subject suffering from or susceptible to a microbiome-dysfunction-associated disease, disorder, or condition) each comprise a clustered regularly interspaced short palindromic repeats (CRISPR) spacer that targets the one or more bacteriophages. In some embodiments, therapeutic bacteria exposed to a subject in need thereof (e.g., a subject suffering from or susceptible to a microbiome-dysfunction-associated disease, disorder, or condition) each comprise at least one or more mutants of bacteriophage receptor(s) on bacterial cell surface.

In some embodiments, a population of such therapeutic bacteria are exposed to a subject suffering from or susceptible to a gut microbiome-dysfunction-associated diseases, disorders, or conditions. Exemplary gut microbiome-dysfunction-associated diseases, disorders, or conditions include, but are not limited to, inflammatory bowel disease (IBD) or irritable bowel syndrome, Crohn's disease, ulcerative colitis, immunotherapy-related colitis. In some such embodiments, therapeutic bacteria exposed to subject suffering from or susceptible to a gut microbiome-dysfunction-associated disease, disorder, or condition are resistant to one or more bacteriophages, which may be or comprise Caudovirales.

In some embodiments, a step of exposing a subject in need thereof to a population of therapeutic bacteria (e.g., ones as described herein) comprises administering to such a subject a composition comprising a population of therapeutic bacteria (e.g., ones as described herein).

In some embodiments, a step of exposing a subject in need thereof to a population of therapeutic bacteria (e.g., ones as described herein) comprises administering to such a subject a composition comprising a nucleic acid sequence for altering the genome of host commensal bacteria in the subject such that the host commensal bacterial are genetically engineered to become resistant to target bacteriophages. In some embodiments, a nucleic acid sequence for altering the genome of host commensal bacteria comprises one or more CRISPR spacers that target one or more target bacteriophages.

In some embodiments, a step of exposing a subject in need thereof to a population of therapeutic bacteria (e.g., ones as described herein) comprises administering to such a subject any one of compositions described herein (including, e.g., a pharmaceutical composition, a cosmetic composition, food or beverage, or a nutritional supplement).

In some embodiments, a population of therapeutic bacteria or a composition described herein (including, e.g., a pharmaceutical composition, a cosmetic composition, food or beverage, or a nutritional supplement) is administered before, during, and/or after treatment with an antimicrobial agent such as an antibiotic. For example, in some embodiments, a population of therapeutic bacteria or a composition described herein can be administered at least about 1 hour, 2 hours, 5 hours, 12 hours, 1 day, 3 days, 1 week, 2 weeks, 1 month, 6 months, or 1 year before and/or after treatment with an antibiotic. In some embodiments, a population of therapeutic bacteria or a composition described herein can be administered at most 1 hour, 2 hours, 5 hours, 12 hours, 1 day, 3 days, 1 week, 2 weeks, 1 month, 6 months, or 1 year before and/or after treatment with an antibiotic.

In some embodiments, a population of therapeutic bacteria or a composition described herein (including, e.g., a pharmaceutical composition, a cosmetic composition, food or beverage, or a nutritional supplement) is administered after treatment with an antibiotic. For example, in some embodiments, a population of therapeutic bacteria or a composition described herein can be administered after the entire antibiotic regimen or course is complete.

In some embodiments, a population of therapeutic bacteria or a composition described herein (including, e.g., a pharmaceutical composition, food or beverage, or a nutritional supplement) is administered before, during, and/or after food intake by a subject. In some embodiments, a population of therapeutic bacteria or a composition described herein is administered with food intake by the subject. In some embodiments, a population of therapeutic bacteria or a composition described herein is administered with (e.g., simultaneously) with food intake.

In some embodiments, a population of therapeutic bacteria or a composition described herein (including, e.g., a pharmaceutical composition, food or beverage, or a nutritional supplement) is administered before food intake by a subject. In some embodiments, a population of therapeutic bacteria or a composition described herein may be more effective or potent at treating a bacterial condition (e.g., a bacterial condition associated with microbiome dysfunction) when administered before food intake. For example, in some embodiments, a population of therapeutic bacteria or a composition described herein can be administered at least about 1 minute, about 2 minutes, about 3 minutes, about 5 minutes, about 10 minutes, about 15 minutes, about 30 minutes, about 45 minutes, about 1 hour, about 2 hours, about 3 hours, about 4 hours, about 5 hours, about 6 hours, about 7 hours, about 8 hours, about 9 hours, about 10 hours, about 12 hours, or about 1 day before food intake by a subject. In some embodiments, a population of therapeutic bacteria or a composition described herein can be administered at most about 1 minute, about 2 minutes, about 3 minutes, about 5 minutes, about 10 minutes, about 15 minutes, about 30 minutes, about 45 minutes, about 1 hour, about 2 hours, about 3 hours, about 4 hours, about 5 hours, about 6 hours, about 7 hours, about 8 hours, about 9 hours, about 10 hours, about 12 hours, or about 1 day before food intake by a subject.

In some embodiments, a population of therapeutic bacteria or a composition described herein (including, e.g., a pharmaceutical composition, food or beverage, or a nutritional supplement) is administered after food intake by the subject. In some embodiments, a population of therapeutic bacteria or a composition described herein is more effective or potent at treating a bacterial condition (e.g., a bacterial condition associated with microbiome dysfunction) when administered after food intake. For example, in some embodiments, a population of therapeutic bacteria or a composition described herein can be administered at least about 1 minute, 2 minutes, 3 minutes, 5 minutes, 10 minutes, 15 minutes, 30 minutes, 45 minutes, 1 hour, 2 hours, 3 hours, 5 hours, 10 hours, 12 hours, or 1 day after food intake by a subject. In some embodiments, a population of therapeutic bacteria or a composition described herein can be administered at most about 1 minute, 2 minutes, 3 minutes, 5 minutes, 10 minutes, 15 minutes, 30 minutes, 45 minutes, 1 hour, 2 hours, 3 hours, 5 hours, 10 hours, 12 hours, or 1 day after food intake by a subject.

Multiple genera or species of therapeutic bacteria can be administered in any order or simultaneously. If simultaneously, multiple genera or species of therapeutic bacteria can be provided in a single, unified form, or in multiple forms, for example, as multiple separate pills. In some embodiments, multiple genera or species of therapeutic bacteria can be packed together or separately, in a single package or in a plurality of packages. In some embodiments, one or all of genera or species of therapeutic bacteria can be given in multiple doses. If not simultaneous, the timing between the multiple doses may vary to as much as about a month.

Compositions described herein can be administered before, during, or after the occurrence of a disease or condition associated with microbiome dysfunction, and the timing of administering such compositions described herein can vary. For example, in some embodiments, compositions described herein can be used as a prophylactic and can be administered continuously to subjects with a propensity to conditions or diseases associated with microbiome dysfunction in order to lessen a likelihood of the occurrence of such diseases or conditions. In some embodiments, compositions described herein can be administered to a subject during or as soon as possible after the onset of the symptoms. In some embodiments, administration of compositions described herein (e.g., comprising therapeutic bacteria) can be initiated within the first 48 hours of the onset of one or more symptoms, within the first 24 hours of the onset of one or more symptoms, within the first 6 hours of the onset of one or more symptoms, or within 3 hours of the onset of one or more symptoms. In some embodiments, initial administration can be via any route practical, such as by any route described herein using composition described herein. In some embodiments, a composition described herein (e.g., comprising therapeutic bacteria) can be administered as soon as is practicable after the onset of a disease or condition associated with microbiome dysfunction is detected or suspected, and for a length of time appropriate for the treatment of such a disease or condition, such as, for example, from about 1 month to about 3 months. The length of treatment can vary for each subject.

Compositions and/or methods described herein can be useful for treatment and/or prophylaxis of a microbiome-dysfunction-associated disease, disorder, or condition. In some embodiments, compositions and/or methods described herein are applicable to animals in general, in particular humans and economically significant domestic animals.

In some embodiments, a microbiome-dysfunction-associated disease, disorder, or condition that may utilize compositions and/or methods described herein is a chronic disorder associated with the presence of abnormal enteric microflora. Such disorders include but are not limited to those conditions in the following categories:

gastro-intestinal disorders including irritable bowel syndrome or spastic colon, functional bowel disease (FBD), including constipation predominant FBD, pain predominant FBD, upper abdominal FBD, non-ulcer dyspepsia (NUD), gastro-oesophageal reflux, inflammatory bowel disease including Crohn's disease, ulcerative colitis, indeterminate colitis, collagenous colitis, microscopic colitis, chronic Clostridium difficile infection, pseudomembranous colitis, mucous colitis, antibiotic associated colitis, idiopathic or simple constipation, diverticular disease, AIDS enteropathy, small bowel bacterial overgrowth, coeliac disease, polyposis coli, colonic polyps, chronic idiopathic pseudo obstructive syndrome;
chronic gut infections with specific pathogens including bacteria, viruses, fungi and protozoa;
viral gastrointestinal disorders, including viral gastroenteritis, Norwalk viral gastroenteritis, rotavirus gastroenteritis, AIDS related gastroenteritis;
liver disorders such as primary biliary cirrhosis, primary sclerosing cholangitis, fatty liver or cryptogenic cirrhosis;
rheumatic disorders such as rheumatoid arthritis, non-rheumatoid arthritidies, non-rheumatoid factor positive arthritis, ankylosing spondylitis, Lyme disease, and Reiter's syndrome;
immune mediated disorders such as glomerulonephritis, haemolytic uraemic syndrome, juvenile diabetes mellitus, mixed cryoglobulinaemia, polyarteritis, familial Mediterranean fever, amyloidosis, scleroderma, systemic lupus erythematosus, and Behcets syndrome;
autoimmune disorders including systemic lupus, idiopathic thrombocytopenic purpura, Sjogren's syndrome, haemolytic uremic syndrome or scleroderma;
neurological syndromes such as chronic fatigue syndrome, migraine, multiple sclerosis, amyotrophic lateral sclerosis, myasthenia gravis, Gillain-Barre syndrome, Parkinson's disease, Alzheimer's disease, Chronic Inflammatory Demyelinating Polyneuropathy, and other degenerative disorders;
psychiatric disorders including chronic depression, schizophrenia, psychotic disorders, manic depressive illness;
regressive disorders including Asbergers syndrome, Rett syndrome, attention deficit hyperactivity disorder (ADHD), and attention deficit disorder (ADD);
regressive disorder, autism;
sudden infant death syndrome (SIDS), anorexia nervosa;
dermatological conditions such as, chronic urticaria, acne, dermatitis herpetiformis and vasculitic disorders.

In some embodiments, a microbiome dysfunction-associated disease, disorder, or condition that may utilize compositions and/or methods described herein is a chronic disorder associated with the presence in the gastrointestinal tract of a mammalian host of abnormal or an abnormal distribution of microflora. Exemplary gut microbiome-dysfunction-associated diseases, disorders, or conditions include, but are not limited to, inflammatory bowel disease (IBD) or irritable bowel syndrome, Crohn's disease, ulcerative colitis, immune-related colitis (e.g., immunotherapy patients who have developed colitis). In some embodiments, subjects administered therapeutic bacteria described herein may be previously administered probiotic therapy (including, e.g., a patient who is receiving or has suffered from a failure of probiotic therapy), fecal microbiota transplantation (FMT) (including, e.g., a patient who is receiving or has suffered from a failure of FMT), and/or immunotherapy (e.g., colitis-associated immunotherapy).

In some embodiments, a composition described herein can be administered in combination with another therapy, including, for example, antibiotic therapy, immunotherapy, chemotherapy, radiotherapy, anti-inflammatory agents, anti-viral agents, anti-microbial agents, anti-fungal agents, probiotic therapy, fecal microbiota transplantation, and combinations thereof. In some embodiments, a composition described herein can be administered prior to another therapy (e.g., ones described herein). In some embodiments, a composition described herein can be administered after another therapy (e.g., ones described herein). For example, in some embodiments, a short course of antibiotics may be administered prior to treatment with compositions described herein, for example, to rid tissue-invasive pathogens originating in the bowel lumen. For example, in treatment of Crohn's disease, in some embodiments, anti- tuberculosis therapy may be required for six to twelve weeks before administration of a composition described herein so that the bowel is cleared out and the flora content exchanged for a predetermined flora.

In some embodiments, administration of a composition described herein can be preceded by, for example, colon cleansing methods such as colon irrigation/hydrotherapy, antibiotic therapy, enema, administration of laxatives, dietary supplements, dietary fiber, enzymes, and magnesium.

### EXEMPLIFICATION

### Example 1. In Vitro Co-Cultures of Phages and Bacteria of Microbiome

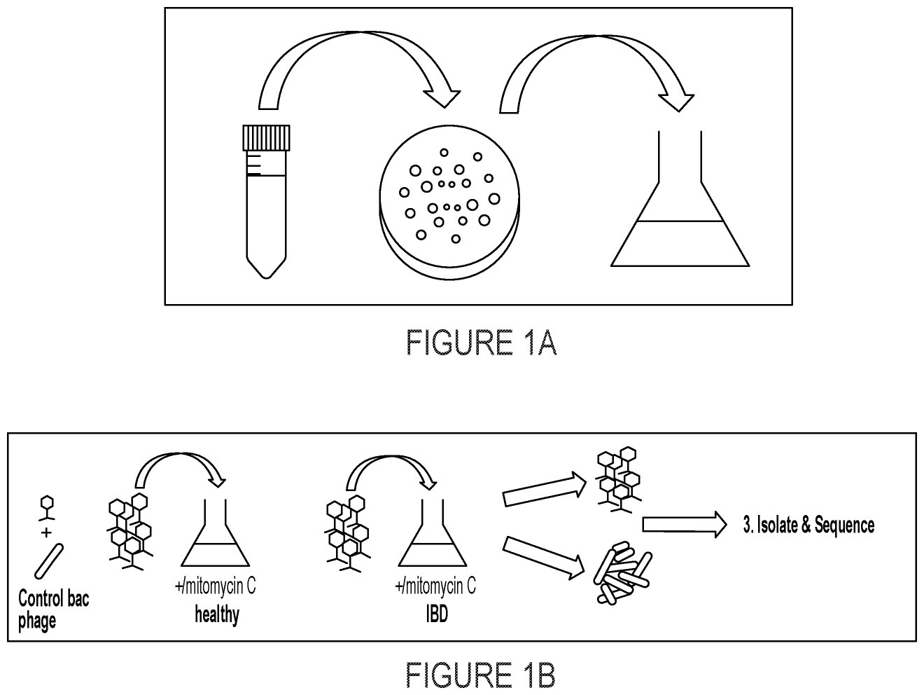

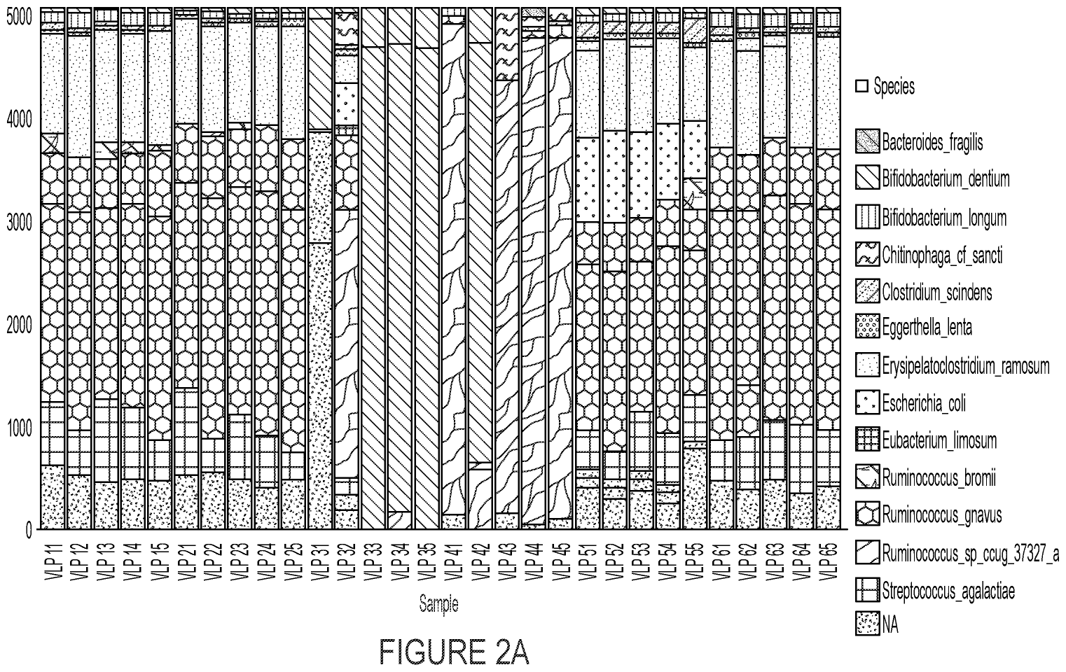

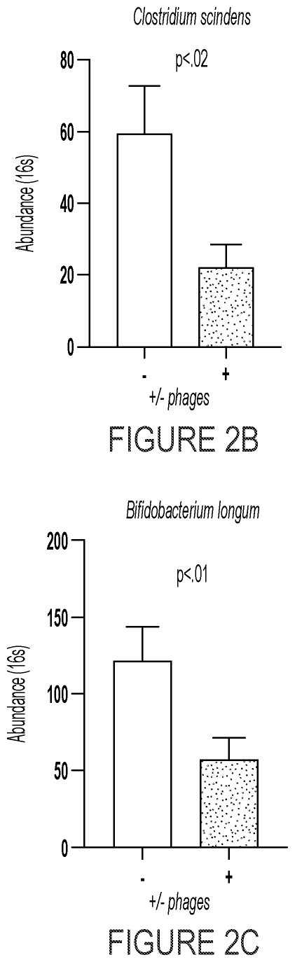

As noted herein, the present disclosure encompasses an insight that bacteriophages (or phages) may be present in certain human organs or tissues (e.g., gut) that deplete beneficial bacterial populations. In light of this insight, the phage fraction was isolated from the fecal sample of a healthy individual and added to a bacterial culture derived from the gut microbiome of the same individual. 16s rRNA sequencing was then performed to characterize changes in the composition of the bacterial community following addition of phage. See, e.g., FIGS. 1A-1B. The present inventor found that addition of phages resulted in the depletion of non-pathogenic commensal bacteria that are known to be important for maintaining health. In particular, it was found that Bifidobacterium longum and Clostridium scindens were depleted after the phage addition (FIGS. 2B-2C). The identification of phages that are able to deplete such bacteria thus confirms the insight provided by the present disclosure that, surprisingly, phages could drive dysbiosis and directly infect and deplete probiotic therapies. This method may be adapted to determine the impact of phages on beneficial bacterial populations in vivo, for example, by isolating the phage fraction from the fecal sample of an individual and adding it to mice which are colonized with the bacterial cultured derived from the gut microbiome of the same or other individual, and characterizing the impact of the phages on the bacterial community.

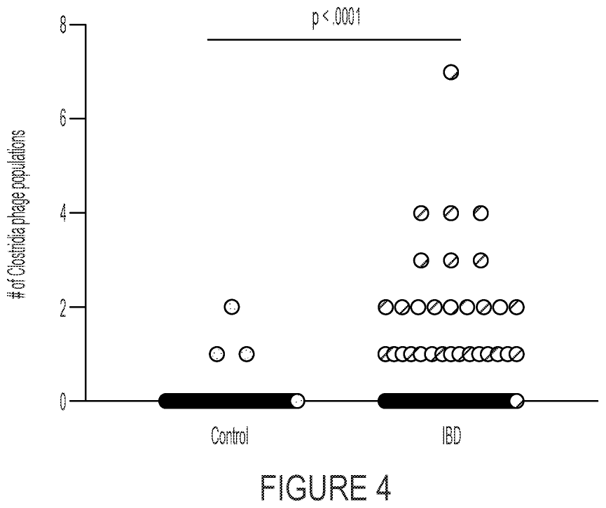

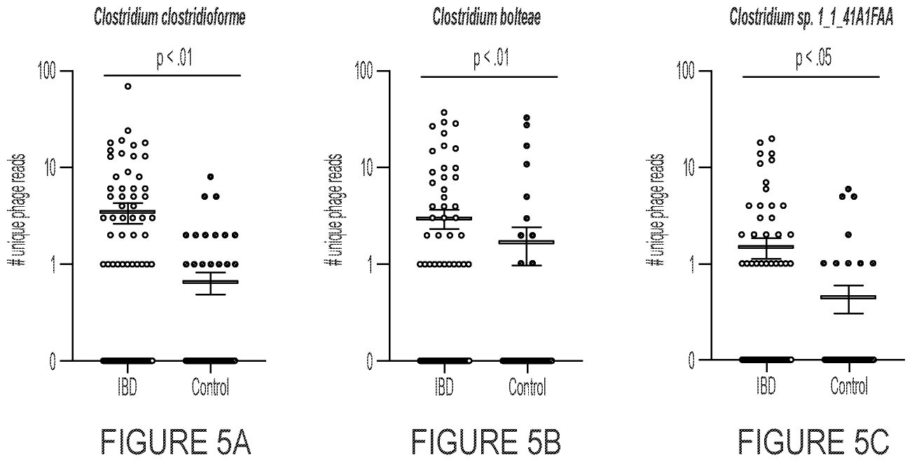

It was also found that phages which infect Clostridia are more prevalent in an IBD patient population than in a healthy population (FIG. 4).

Below is an exemplary list of Clostridia bacteria for which phages that infect these species/strains present in the gut of patients with IBD were identified:

| | |
|---|---|
| Clostridia bacterium | Clostridium sp. MSTE9 |
| UC5.1-1A9 | |
| Clostridium asparagiforme | Clostridium sp. VE202-10 |
| Clostridium cellulosi | Clostridia bacterium UC5.1-2G4 |
| Clostridium bolteae | Clostridia bacterium UC5.1-2H11 |
| Clostridium citroniae | Clostridiales bacterium 1_7_47FAA |
| Clostridium clostridioforme | Clostridiales bacterium JGI |
| | 000176CP_D02 |
| Clostridium indolis | Clostridiales bacterium VE202-03 |
| Clostridium cocleatum | Clostridiales bacterium VE202-06 |
| Clostridium innocuum | Clostridiales bacterium VE202-07 |
| Clostridium lavalense | Clostridiales bacterium VE202-09 |
| Clostridium saccharolyticum | Clostridiales bacterium VE202-15 |
| Clostridium scindens | Clostridiales bacterium VE202-16 |
| Clostridium symbiosum | Clostridiales bacterium VE202-21 |
| Clostridium butyricum | Clostridiales bacterium VE202-26 |
| Clostridia bacterium | Clostridiales bacterium VE202-27 |
| UC5.1-1A9 | |
| Clostridium jeddahense | Clostridiales bacterium VE202-28 |
| Clostridium nigeriense | Clostridiales bacterium VE202-29 |
| Clostridium neonatale | Clostridium sp. C8 |
| Clostridium perfringens | Clostridium sporogenes |
| Clostridium phoceensis | Clostridium tyrobutyricum |
| Clostridium sp. 1_1_41A1FAA | Clostridium sp. VE202-10 |
| Clostridium sp. 316002/08 | Lachnoclostridium sp. An131 |
| Clostridium sp. 7_3_54FAA | Lachnoclostridium sp. YL32 |
| Clostridium sp. ATCC BAA-442 | Lachnospiraceae bacterium |
| | 3_1_57FAA_CT1 |
| Clostridium sp. C105KSO14 | Lachnospiraceae bacterium |
| | 5_1_57FAA |
| Clostridium sp. CL-6 | Lachnospiraceae bacterium |
| | 6_1_63FAA |
| Clostridium sp. D5 | Lachnospiraceae bacterium A4 |
| Clostridium sp. FS41 | Lachnospiraceae bacterium |
| | DJF_VP30 |
| Clostridium sp. HGF2 | Lachnospiraceae bacterium VE202-23 |
| Clostridium sp. IODB-03 | Lachnospiraceae bacterium VE202-12 |
| Clostridium sp. KLE 1755 | Clostridium acetireducens |
| Clostridium sp. L2-50 | Clostridium collagenovorans |
| Clostridium sp. M62/1 | |

### Exemplary Method:

Fecal sample was homogenized in 12 ml of sterile 20% glycerol/1X PBS in a 50 ml conical and serial dilutions were plated on 0.1% mucin BHI agar. Plates were scraped, diluted in PBS. 5 ul of diluted sample was added to 5m1 of 0.1% BHI media. 2 ul of isolated virus-like particles (VLPs) from the same sample was added to plate culture. As a positive control, E.coli and T7 phage were added to a subset of cultures. Cultures were grown anaerobically for 72 hours. Samples were then spun down and prepped for sequencing.

### Example 2. Computational Approach for Identification of Phages and Their Bacterial Hosts Present in Patients with Inflammatory Bowel Disease

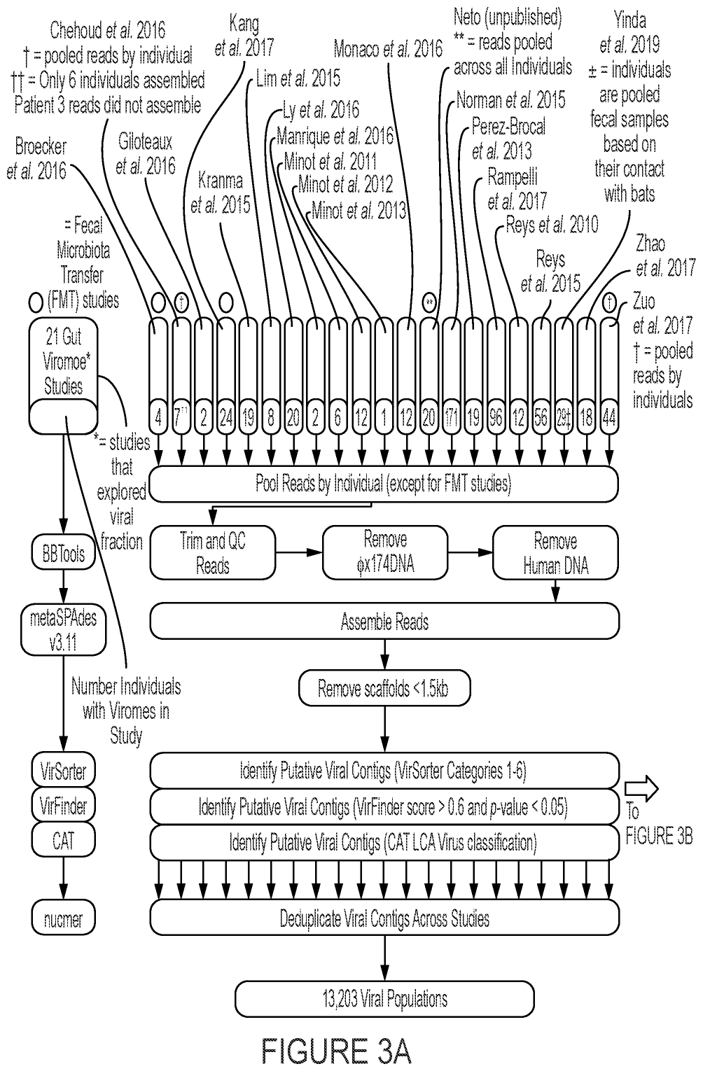

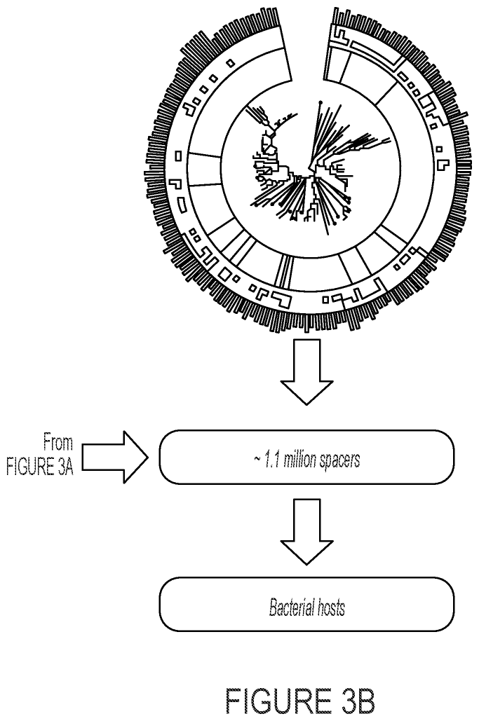

A CRISPR-based approach can be used to predict bacterial targets or hosts of phages present across individuals. A curated list of CRISPR spacer sequences extracted from a wide range of gut bacteria was analyzed. Gregory et al. “The human gut virome database” bioRxiv 655910 (May 2019). The presence of a given CRISPR spacer sequence in a bacterial population provides evidence that a phage containing that sequence infected that bacteria. Further, putative non-pathogenic commensal bacterial hosts for phages and/or phages present in individuals that attack non-pathogenic commensal bacteria can be identified by matching viral sequences to CRISPR spacer sequences from known bacterial hosts. See, e.g., FIG. 3. Such information can be used to develop phage-resistant non-pathogenic commensal bacteria.

### Example 3. Engineering Exemplary Phage-Resistant Non-Pathogenic Commensal Bacteria

Engineered phage-resistant non-pathogenic commensal bacteria to block infectious gut phages in vitro.

As described in Examples 1 and 2, data presented herein suggest that infectious phages (e.g., present in the gut) deplete bacteria in the microbiome, and that phage that target beneficial bacteria are significantly more prevalent in patients with IBD. Building on such findings, commensal bacteria are engineered to be resistant to infectious phages that target gut bacteria. The efficacy of these bacteria in resisting phage proliferation in vitro and in vivo are then assessed.

Phage-resistant bacteria are engineered based on the identification of bacterial hosts for phages and corresponding phage sequence information. In some embodiments, one or more sequences matching one or more identified phages are introduced into CRISPR (e.g., CRISPR-Cas) loci of a non-pathogenic commensal bacterial strain. Methods to modulate resistance in a cell against a target nucleic acid are known in the art. See, e.g., U.S. Pat. No. 10,066,233. One skilled in the art, reading the present disclosure, will thus understand that such methods and other methods known in the art (including, e.g., CRISPR-Cas technologies) can be useful for engineering therapeutic compositions of non-pathogenic and commensal bacteria which are resistant to one or more bacteriophages (e.g., as described herein). In some embodiments, bacteriophage-resistant non-pathogenic and commensal bacteria can be engineered by inserting one or more target phage sequences as CRISPR spacer(s) within a CRISPR array (e.g., an endogenous CRISPR array or an exogenous/heterologous CRISPR array) present in a non-pathogenic and commensal bacterial strain of interest. For example, in some embodiments, a phage which infects a given non-pathogenic and commensal bacterial strain of interest can be identified using techniques described in Examples 1 and 2. In some embodiments, a CRISPR-Cas locus sequence in a non-pathogenic and commensal bacterial strain can be identified by sequencing a relevant portion of the genome (including, e.g., the entire genome in some embodiments) of the bacterial strain. In some such embodiments, a protospacer-adjacent motif (PAM) sequence recognized by an identified CRISPR-Cas system is characterized. See, e.g., Mendoza and Trinh, Biotechnol J. (2018) 13:e1700595; and Gleditzsch et al., RNA Biol. (2019) 16:504-517. A suitable spacer sequence is typically selected from the genome of a phage that is identified to infect a given non-pathogenic and commensal bacterial strain of interest. In some embodiments, a suitable spacer sequence is or comprises a conserved sequence. In some embodiments, such a conserved sequence may be present in a phage gene (e.g., a phage gene that is present in different genus, species, and/or strains of phages). In some embodiments, a suitable spacer sequence can be or comprise a sequence that is up to 8 nucleotides upstream of the PAM sequence. A CRISPR unit comprising or consisting of a suitable spacer sequence flanked by two repeating elements (e.g., CRISPR repeats) is then introduced into a non-pathogenic and commensal bacterial strain of interest. In some embodiments, a spacer sequence can be introduced into an endogenous CRISPR-Cas array of a non-pathogenic and commensal bacterium. In some embodiments, spacer sequence(s) can be introduced in trans, on a synthetic array that is compatible with an endogenous Cas machinery (e.g. elsewhere on the genome, or on a plasmid). In some embodiments, spacer sequence(s) can be introduced in trans in a synthetic array which is co-delivered with exogenous cas genes.

In some embodiments, multiple spacer sequences can be used to target each distinct phage population. Accordingly, in some embodiments, at least one or more (including, e.g., at least two or more) spacer sequences targeting a single bacteriophage population are introduced into CRISPR loci of a non-pathogenic commensal bacterium. Spacer diversity can enhance protection against phage infection. Thus, in some such embodiments, at least two or more spacer sequences are identical, while in some such embodiments, at least two or more spacer sequences are distinct.

In some embodiments, multiple (e.g., at least two, at least three, or more) spacer sequences targeting different phage populations or families can be introduced into a single non-pathogenic and commensal bacterium. Such engineered non-pathogenic and commensal bacterium can be useful for providing resistance against multiple different phage populations or families.

In some embodiments, a spacer sequence introduced into CRISPR loci of a non-pathogenic commensal bacterium is 20-50 bp long. In some embodiments, a spacer sequence introduced into CRISPR loci (e.g., CRISPR-Cas loci) of a non-pathogenic commensal bacterium is selected from the ones identified to match CRISPR spacer sequences identified in given bacterial hosts, and/or can also be selected from phage sequences. In some embodiments, sequences that are conserved among various phage populations (or across populations) are chosen.

In some embodiments, non-pathogenic commensal bacteria as described herein may play crucial roles in the maintenance of health and/or be useful as therapies to treat a number of chronic conditions.

Tools for genetic engineering are well known in the art; one skilled in the art will thus understand that such tools can be useful for engineering non-pathogenic commensal bacteria to become phage-resistant as described herein. As will be understood by those skilled in the art, in some embodiments, CRISPR/Cas9 based genome editing tools, which have the advantage of efficient marker-less gene editing, may be used to engineer phage-resistant non-pathogenic commensal bacteria. See, e.g., Barrangou et al. Science (2007) 315:1709-1712; Deveau et al. Journal of Bacteriology (2008) 190: 1390-1400; Barrangou and Marraffini, Mol Cell (2014) 54:234-244; Crawley et al. CRISPR J. (2018) 1:171-181; Crawley et al. Scientific Reports (2018) 8:11544; and Hidalgo-Cantabrana et al. PNAS (2019) 116: 15774-15783 for information relating to methods of CRISPR-Cas identification and/or characterization, selection and/or engineering of spacers, and/or genome editing using CRISPR-Cas system. The contents of each of the references cited herein are incorporated herein by reference in their entireties for purposes described herein.

Engineered phage-resistant non-pathogenic commensal bacteria can be assessed in vitro using methods known in the art. For example, in some embodiments, engineered phage-resistant non-pathogenic commensal bacteria can be assessed in vitro by measuring the survival of such engineered bacteria in the presence of infectious phage. Infectious phage can be collected from a viral fraction of fecal samples of patients in which infectious phages are identified to be present. The collected viral fraction is then filter-sterilized. Phages from the filtered viral fraction are added to a culture (e.g., monoculture) of (i) one or more engineered phage-resistant non-pathogenic commensal bacterial strain or (ii) a wild-type (WT) strain as a control. Growth of bacterial strains in the presence of phages is assessed, for example, using plaque assays.

Growth in a monoculture may be different from growth in a mixed bacterial culture of bacteria, e.g., microbiome from the gut. Accordingly, in some embodiments, survival of engineered phage-resistant non-pathogenic commensal bacteria can be assessed in a mixed bacterial culture in the presence of infectious phage.

### Example 4. Engineering Exemplary Phage-Resistant Commensal Bacteria In Vivo

To assess ability of engineered phage resistant bacteria to combat phage predation and facilitate colonization in vivo, human microbiota-associated (HMA) mouse models are used. Mice pre-treated with antibiotics are colonized with fecal samples in which infectious phages are identified against target non-pathogenic commensal bacteria hosts, resulting in robust colonization of mice with human microbiota. Engineered phage-resistant non-pathogenic commensal bacteria (e.g., ones that demonstrate robust efficacy in in vitro assays, for example, as described in Example 3 above) or WT strains are then added to HMA mice. The composition of the overall microbiota and engineered phage-resistant non-pathogenic commensal bacterial strains can be monitored via 16s rRNA sequencing and qPCR, respectively, at weekly time intervals for a certain period of time (e.g., for a period of 28 days).

In some embodiments, engineered phage resistant bacteria are assessed in specific pathogen free (SPF) mouse models. For example, (i) phages targeting non-pathogenic and commensal bacteria of interest and (ii) therapeutic compositions comprising or consisting of phage-resistant non-pathogenic and commensal bacteria (e.g., as described herein) are introduced to the gut of SPF mice. Levels of phages and phage resistant bacterial strains are assessed, for example, with qPCR using primers complementary to the engineered bacterial strain.

Phages from mouse fecal samples are also isolated and sequencing on such samples are performed to assess whether replication of target phages is reduced in the presence of phage-resistant non-pathogenic commensal bacterial strains.

### Example 5. Using Exemplary Phage-Resistant Clostridium to Treat Inflammatory Bowel Disease (IBD).

As discussed above, the present inventor found that phages that attack bacteria in the Clostridia class are significantly more prevalent in patients with IBD. Such Clostridia bacteria primarily include a number of species that are shown to have anti-inflammatory benefits. For example, it was previously reported that many of such Clostridium species promote the induction of colonic regulatory T (Treg) cells in mice, and oral inoculation of a mixture of 17 Clostridia strains attenuated disease in mouse models of colitis. The present finding indicates that a phage-driven reduction in anti-inflammatory Clostridium species can promote or contribute to the development of inflammation and IBD disease in susceptible individuals.

To assess ability of phage resistant bacteria to treat dysbiosis and disease in mouse models of IBD, bacterial and viral fractions of fecal samples from healthy individuals or IBD patients are separated. Mice pre-treated with antibiotics are then colonized with the bacterial fraction from a healthy individual, for example, via oral gavage. Mice are then orally inoculated with phages isolated from healthy individuals or individuals with IBD, and one or more phage-resistant or WT non-pathogenic commensal bacteria strains. Levels of the phage-resistant and WT bacteria are tracked over time, for example, using qPCR, and disease status may be monitored via body weight, measurements of occult blood, and/or inflammatory markers.

Efficacy of phage-resistant non-pathogenic commensal bacteria can also be assessed using mice that are colonized directly with a fecal sample of one or more individuals with IBD. WT and phage-resistant non-pathogenic commensal bacteria are administered and the levels of such bacteria are tracked over time, then disease status may be monitored via body weight, measurements of occult blood, and/or inflammatory markers.

### Example 6. Engineering Phage-Resistant Bifidobacterium longum

In some embodiments, Bifidobacterium longum present in a human gut can harbor Type I-C CRISPR systems. In some embodiments, spacer sequences that match target phages present in the gut that deplete these bacteria can be introduced into these CRISPR arrays to confer resistance. Phage-resistant bacteria can be assessed for improved growth in the presence of target phages in vitro and in vivo.

### Example 7. Engineering Phage-Resistant Lactobacillus

Several commensal species of Lactobacillus have been shown to be important for human health, including, but not limited to, Lactobacillus gasseei, Lactobacillus crispatus, and Lactobacillus acidophilus. In some embodiments, phage resistant strains of Lactobacillus can be engineered to resist phages present in a human gut microbiome using methods described in Example 3. Therapeutic compositions comprising such engineered, phage-resistant strains can be useful for administration to affected individuals to treat a microbiome-dysfunction associated disease.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

Those skilled in the art will recognize, or be able to ascertain using no more than routine experimentation, many equivalents to the specific embodiments of the invention described herein. It is to be understood that the invention encompasses all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. Further, it should also be understood that any embodiment or aspect of the invention can be explicitly excluded from the claims, regardless of whether the specific exclusion is recited in the specification. The scope of the present invention is not intended to be limited to the above Description, but rather is as set forth in the claims that follow.

What is claimed is:

1. A method comprising a step of:
exposing a subject suffering from or susceptible to a microbiome-dysfunction-associated disease, disorder, or condition, to a population of therapeutic bacteria that (i) are non-pathogenic and commensal in the subject and (ii) are resistant to one or more target bacteriophages.

2. The method of claim 1, wherein the therapeutic bacteria each comprise at least one clustered regularly interspaced short palindromic repeats (CRISPR) spacer that targets the one or more target bacteriophages.

3. The method of claim 1 or 2, wherein the therapeutic bacteria each comprise a mutation in one or more receptors of the therapeutic bacteria for a target bacteriophage-receptor binding protein.

4. The method of claim 2 or 3, wherein the microbiome is a gut microbiome.

5. The method of claim 4, wherein the gut microbiome-dysfunction-associated disease, disorder, or condition is inflammatory bowel disease (IBD) or irritable bowel syndrome.

6. The method of claim 4, wherein the gut microbiome-dysfunction-associated disease, disorder, or condition is Crohn's disease.

7. The method of claim 4, wherein the gut microbiome-dysfunction-associated disease, disorder, or condition is ulcerative colitis.

8. The method of claim 4, wherein the gut microbiome-dysfunction-associated disease, disorder, or condition is immunotherapy-related colitis.

9. The method of any one of claim 1-8, wherein the one or more target bacteriophages are associated with the microbiome-dysfunction-associated disease.

10. The method of any one of claims 1-9, wherein the one or more bacteriophages are temperate or non-lytic bacteriophages.

11. The method of any one of claims 1-10, wherein the one or more bacteriophages are or comprises Caudovirales.

12. The method of any one of claims 1-11, wherein the therapeutic bacteria are or comprise Bacteroides, Bifidobacterium, Clostridium, Escherichia, Lactobacillus, Lactoccucs, or combinations thereof

13. The method of any one of claims 1-12, wherein the subject has been administered probiotic therapy, fecal microbiota transplantation (FMT), and/or immunotherapy (e.g., colitis-associated immunotherapy).

14. The method of any one of claims 1-13, wherein the step of exposing comprises administering to the subject a composition comprising the population of the therapeutic bacteria.

15. The method of any one of claims 1-14, wherein the step of exposing comprises administering to the subject a composition comprising a nucleic acid sequence encoding the CRISPR spacer, wherein the composition is delivered to host commensal bacteria of the subject to produce the therapeutic microbe.

16. The method of claim 15, wherein the nucleic acid is delivered by a recombinant bacteriophage.

17. The method of claim 15, wherein the nucleic acid is delivered by a vector.

18. A therapeutic composition comprising an engineered population of therapeutic bacteria that (i) are non-pathogenic and commensal in a subject to be administered; and (ii) are resistant to one or more target bacteriophages.

19. The therapeutic composition of claim 18, wherein the therapeutic bacteria each comprise a clustered regularly interspaced short palindromic repeats (CRISPR) spacer that targets the one or more target bacteriophages.

20. The therapeutic composition of claim 18, wherein the therapeutic bacteria are genetically engineered to express a CRISPR spacer that targets the one or more target bacteriophages.

21. The therapeutic composition of any one of claims 18-20, wherein the therapeutic bacteria are or comprise Bacteroides, Bifidobacterium, Clostridium, Escherichia, Lactobacillus, Lactococcus, Akkermansia, or combinations thereof.

22. The therapeutic composition of any one of claims 18-21, wherein one or more receptors of the therapeutic bacteria for a target bacteriophage-receptor binding protein are mutated such that the therapeutic bacteria are resistant to the target bacteriophage.
